(19) United States
(12) Patent Application Publication
(10) Pub. No.: US 2007/0022482 A1
(43) Pub. Date: Jan. 25, 2007

(54) High-throughput functional analysis of gene expression

(76) Inventors: Craig E. Eckfeldt, Minneapolis, MN (US); Eric M. Mendenhall, Minneapolis, MN (US); Stephen C. Ekker, St. Paul, MN (US); Catherine M. Verfaillie, St. Paul, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/452,081

(22) Filed: Jun. 13, 2006

Related U.S. Application Data
(60) Provisional application No. 60/690,089, filed on Jun. 13, 2005.

(51) Int. Cl. A01K 67/027; C12Q 1/68; G06F 19/00

(52) U.S. Cl. 800/8; 702/20; 435/6

(57) ABSTRACT

The invention is directed to an in vivo functional genomics screen for extracting functional information from a global gene expression profiling dataset in a high-throughput manner.

### REFERENCE TO THE RELATED APPLICATIONS

This application claims priority to U.S. Provisional Application Ser. No. 60/690,089, filed Jun. 13, 2005, the contents of the provisional application is incorporated herein by reference in its entirety.

### FEDERALLY SPONSORED RESEARCH OR DEVELOPMENT

This research was supported by the National Cancer Institute Program Project Grant (CA065493) and National Institute General Medical Sciences R01 (GM63904). The government may have certain rights to this invention.

### BACKGROUND OF THE INVENTION

Hematopoiesis is the process by which hematopoietic stem cells (HSCs) give rise to all hematopoietic lineages during the lifetime of an individual. To sustain life-long hematopoiesis, HSC must self-renew to maintain or expand the HSC pool [1], and they must differentiate to form committed hematopoietic progenitor cells (HPCs) that progressively lose self-renewal potential and become increasingly restricted in their lineage potential. A combination of extrinsic and intrinsic signals are thought to converge to regulate HSC differentiation versus self-renewal decisions, but the molecular mechanisms that regulate these processes are poorly understood [2].

A multitude of cytokines have been cloned that affect HSCs and HPCs; however, to date none of these, alone or in combination, can induce the symmetrical, self-renewing HSC division in vitro that is needed for HSC expansion. Recently, several novel regulators of HSC fate decisions have been identified. For instance, overexpression of HoxB4 (GeneID: 15412) results in expansion of murine and human HSCs with an increased competitive repopulation potential [3,4,5]; novel extrinsic regulators implicated in self-renewal of HSCs include Notch [6], Wnt [7,8], and the morphogens, sonic hedgehog (Shh) (GeneID: 6469) [9] and bone morphogenetic protein (BMP)-4 (GeneID: 652) [9]. While the discovery of these novel regulators provides credence to the hypothesis that extrinsic and intrinsic signals can influence HSC fate, a more global gene and/or protein expression analysis of human HSC should provide additional insight into pathways that support HSC self-renewal.

### SUMMARY OF THE INVENTION

The invention is directed to an in vivo functional genomics screen for extracting functional information for a global gene expression profiling dataset in a high-throughput manner. A global gene expression profiling dataset can be obtained from, for example, differential expression analysis, such as that obtained from gene microarray analysis or quantitative RT-PCR analysis. The invention also provides use of the discovered genes and/or function of the genes. For example, the genes that were discovered to play a role in hematopoiesis can be manipulated to increase or decrease their expression in stem cells (for example, to expand stem cells or differentiate them) and/or in the treatment of disorders (e.g., blood disorders) or diseases (e.g., cancer), for example, via gene therapy.

One embodiment of the invention provides an in vivo method of assigning function to a gene (the function of a gene generally refers to the function of the protein that the gene codes, e.g., a function/role in hematopoiesis) comprising: a) providing a gene expression profiling dataset; b) identifying (e.g., by comparison to sequences present in a database) at least one ortholog in an animal model for at least one gene of an unknown function from the dataset; c) altering expression (either increasing or decreasing (including preventing) expression of a gene or its protein product) of the ortholog in the animal model; d) detecting one or more changes in the animal model due to alteration of the expression; and e) correlating the one or more changes in the animal model with the function of the gene. Another embodiment further comprises compiling a functional profile (e.g., correlating a function with one or more of the genes provided in the gene expression profile) comprising repeating steps b)-e) until two or more genes from the dataset having unknown function are associated with a function.

In one embodiment, the gene expression profiling dataset is obtained from differential gene expression analysis, including, but not limited to, gene microarray analysis or quantitative PCR analysis.

In one embodiment, the animal model is a mouse, rat, zebrafish or xenopus animal model. In another embodiment, the animal model is an embryonic cell model.

In one embodiment, the expression of the ortholog in the animal is decreased, such as by the use of antisense oligonucleotides. In one embodiment, the antisense oligonucleotides are morpholino antisense oligonucleotides.

In one embodiment, the one or more changes detected are phenotypic (e.g., any detectable characteristic of an organism (i.e., structural, biochemical, physiological (including, for example, a decrease or increase in blood production or a decrease or increase in the amount of a transcript or protein produced, such as a transcription factor or a protein which is cell marker) and behavioral). In another embodiment, the phenotypic change is an alteration in blood cell production or transcription factor expression.

One embodiment provides increased (as compared to an animal (of the same species) without increased (e.g., overexpression) ortholog gene expression) ortholog expression in the animal model.

### BRIEF DESCRIPTION OF THE DRAWINGS

FIG. 1. Fluorescence Activated Cell Sorting (FACS) and gene expression analysis. Umbilical cord blood or adult bone marrow CD34⁺CD33⁻CD38⁻Rho^{lo}c-kit⁺ (Rho^{lo}; stem cell enriched) and CD34⁺CD33⁻CD38⁻Rho^{hi} (Rho^{hi}; stem cell depleted) cell populations were sorted for subsequent global gene expression profiling. Total RNA was isolated from Rho^{lo} and Rho^{hi} cell populations prior to linear amplification and labeling for hybridization to the Affymetrix® HG-U133 GeneChip™ set (˜45,000 probe sets) and subsequent expression data normalization and analysis.

FIG. 2 (A-B). Functional genomics screen in zebrafish. (A) Overview of morpholino antisense oligonucleotide technology. (B)The hematopoietic function of differential expressed candidate genes was determined by injecting morpholino antisense oligonucleotides (morpholinos or MOs) into one to two cell embryos from gata1:DsRed Tg zebrafish to disrupt gene expression. Injected embryos were scored at 30-48 hpf for the presence of DsRed⁺ blood cells by fluorescence microscopy. Subsequently, MO-targeted embryos with gross hematopoietic defects were analyzed for the expression of the early hematopoietic markers gata1 and scl by whole-mount in situ hybridization, and the late hematopoietic markers hbae1 and lcp1 by quantitative RT-PCR (Q-RT-PCR).

FIG. 3. Representative hematopoietic phenotypes observed in MO-targeted zebrafish. Fluorescence microscopic images of gata1:DsRed Tg zebrafish embryos display the hematopoietic phenotypes observed for six MO-targeted zebrafish embryos compared to an un-injected control. Phenotypes shown are representative of >70% of injected embryos at 48 hours post-fertilization (>3 experiments of n>40 embryos). Hematopoietic defects were quantified by quantitative RT-PCR (Q-RT-PCR) for the expression of erythroid-specific hbae1 and myeloid-specific lcp1 transcripts in MO-targeted embryos relative to uninjected clutchmate controls (>3 experiments of n=5 embryos; *p<0.05).

FIG. 4(A-D). spry4 plays a role in normal hematopoietic development in zebrafish embryos. (A) The observed frequency of hematopoietic defects in gata1:DsRed transgenic (Tg) zebrafish embryos are indicated for two independent spry4-targeted MOs (MO1 and MO2; black bars), a 4-base mismatched control MO (MM MO; no bar indicates a 0% frequency), a low dose injection of MO1 and MO2 individually and in combination (gray bars), and MO2 co-injected with a human SPRY1 DNA expression vector or a GFP control (white bars) (error bars=standard deviation of the mean, *p<0.05, **p≦0.01). (B) Representative pictures of the phenotypes seen with spry4 MO1 at 48 hours using fli1:eGFP/gata1:DsRed double Tg zebrafish embryos that have eGFP⁺ vascular endothelial cells and DsRed⁺ erythroid cells. The embryos display a more drastic reduction in DsRed⁺ blood cells with some blood pooling and pericardial edema at higher MO doses (++MO) without major vasculature defects. The gata1:DsRed and fli1:eGFP Tg images are representative of 3 experiments of n>40 embryos each. (C) Quantitative RT-PCR (Q-RT-PCR) analysis of hbae1 and lcp1 transcripts in spry4 MO1, control mismatch MO and a gata1 MO injected zebrafish at 48 hpf (*p<0.05). (D) Injection of 30 pg of human SPRY1 DNA expression vector resulted in an expansion of DsRed⁺ hematopoietic cells in the posterior ICM in >50% of successfully injected embryos at 32 hpf (3 experiments of n=30), and a representative bright-field image is pictured with the fluorescence micrograph overlayed (left). Q-RT-PCR analysis of hbae1 and lcp1 transcripts in SPRY1 overexpressing (OE) embryos relative to uninjected clutchmate controls (3 experiments of n=5 embryos) (right).

FIG. 5(A-E). spry4 plays a role in early hematopoietic development, but generally not mesodermal commitment, in developing zebrafish embryos. (A) Expression of the early hematopoietic marker, sc1, was reduced at the four somite stage (arrow) and virtually absent at the 20 somite stage in spry4^{MO} embryos compared to uninjected controls. (B) The more mature hematopoietic marker, gata1, was also reduced in spry4^{MO} compared to uninjected zebrafish embryos at 20 somites. (C,D) In contrast to hematopoietic genes, the mesodermal-specific flk1 (vasculature) transcripts were expressed at similar levels to uninjected controls at 26 hpf and myod (muscle) had a slight defect (arrowhead) at 10 hpf, while expression was similar to controls at 26 hpf. All Images are representative of ≧2 experiments of n≧8 embryos.

FIG. 6. Frequency of Myeloid-Lymphoid Initiating Cells (ML-IC) in UCB Rho^{lo} and Rho^{hi} cell populations. ML-ICs are highly enriched in Rho^{lo} (white bar) compared to Rho^{hi} (black bar) cells from UCB (n=3, *p<0.05).

FIG. 7. Confirmation of differential gene expression using quantitative RT-PCR (Q-RT-PCR). The Rho^{lo} to Rho^{hi} fold change for DLK1, ABCB1, BMP6, HELLS, CDC25A, MAFB and S100A8 was determined using Affymetrix® GeneChip™ analysis (grey bars) and Q-RT-PCR (black bars) to confirm the fidelity of the microarray results for (A) adult bone marrow (n≧2) and (B) umbilical cord blood (n24).

FIG. 8. Gene Ontology (GO) classifications of conserved differentially expressed genes. Percentages of gene ontology classifications of the genes differentially expressed between Rho^{lo} and Rho^{hi} cells from both umbilical cord blood and adult bone marrow (p<0.05 and fold change>1.5 in either UCB or BM).

FIG. 9. Morpholino sequences for targeted genes (SEQ ID NO: 1-64).

FIG. 10. Complete phenotypic descriptions for the 14 zebrafish morphants with confirmed blood defects.

FIG. 11. Q-RT-PCR primer sequences (SEQ ID NO:65-88).

FIG. 12. Depicts an overview of human hematopoiesis.

FIG. 13. Depicts MO knock-down of genes which resulted in decreased blood in zebrafish.

### DETAILED DESCRIPTION OF THE INVENTION

Definitions

As used herein, the terms below are defined by the following meanings:

As used herein, the term “ortholog” refers to genes in different species which evolved from a common ancestral gene. Due to their separation following a speciation event, orthologs may diverge, but usually have similarity at the sequence and structure levels. Orthologous genes are inherited through vertical descent from a common ancestor. These genes may arise from a common ancestral gene after speciation has occurred, or they may be present as polymorphic alleles in a population before speciation occurs. Not all orthologs perform the same, or even similar, functions as their counterparts.

As used herein, the phrase “animal model” refers to a non-human animal or embryo (e.g., mouse models, zebrafish models, dog models etc.) with a disease or biological system or activity that is similar to a human condition or system/activity (e.g., the development of blood or the vasculature). The use of animal models allows researchers to investigate disease states or biological processes.

“Genes” are the units of heredity in living organisms. They are encoded in the organism's genetic material (usually DNA or RNA), and control the physical development and behavior of the organism. Genes encode the information necessary to construct the chemicals (proteins etc.) needed for the organism to fuinction. The term “genes” generally refers to the region of DNA (or RNA, in the case of some viruses) that determines the structure of a protein (the coding sequence), together with the region of DNA that controls when and where the protein will be produced (the regulatory sequence).

Differential gene expression analysis/techniques include, but are not limited to, differential screening (e.g., with use of, for example, a phage library), subtractive screening (an RT-PCR based method, such as suppression subtractive hybridization (SSH)), differential display and DNA microarray (e.g., Affymetrix (commercial) or Spotted arrays)). The study of differential gene expression provides biologically information regarding gene expression. For example, the correlation of changes in gene expression with specific changes in physiology can provide information to assign a function to the gene whose expression changed. Differential gene expression techniques are well known in the art.

Antisense oligonucleotides interact with complementary strands of nucleic acids, modifying expression of genes. Antisense oligonucleotide technology is known in the art. Morpholino oligonucleotides are molecules used in antisense technology to block access of other molecules to specific sequences within nucleic acid molecules. They can block access of other molecules to small (˜25 base) regions of ribonucleic acid (RNA). Morpholinos are sometimes referred to as PMO, an acronym for phosphorodiamidate morpholino oligo.

Morpholinos are synthetic molecules which are the product of a redesign of natural nucleic acid structure. Usually 25 bases in length, they bind to complementary sequences of RNA by standard nucleic acid base-pairing. Structurally, the difference between Morpholinos and DNA is that while Morpholinos have standard nucleic acid bases, those bases are bound to morpholine rings instead of deoxyribose rings and linked through phosphorodiamidate groups instead of phosphates. Replacement of anionic phosphates with the uncharged phosphorodiamidate groups eliminates ionization in the usual physiological pH range, so Morpholinos in organisms or cells are uncharged molecules. Morpholinos are not chimeric oligos; the entire backbone of a Morpholino is made from these modified subunits. Morpholinos are most commonly used as single-stranded oligos, though heteroduplexes of a Morpholino strand and a complementary DNA strand may be used in combination with cationic cytosolic delivery reagents.

Morpholinos do not degrade their target RNA molecules, unlike many antisense structural types (e.g., phosphorothioates, siRNA). Instead, Morpholinos act by “steric blocking”, binding to a target sequence within an RNA and simply getting in the way of molecules which might otherwise interact with the RNA.

A gene expression profiling dataset is a preselected set of genes of interest (e.g., genes for which determination of function is desirable); such a dataset can be obtained from, for example, differential gene expression analysis/techniques (e.g., a dataset can be formed of genes that are differentially expressed in one source or combined sources, a dataset can also be comprised of any gene or genes for which the determination of function is desirable).

As used herein, the phrase “expression profiling” refers to differential gene expression analysis/techniques, such as microarray technology. Microarray technology allows for the comparison of gene expression between, for example, normal and diseased (e.g., cancerous) cells or cells which express different cell markers. There are several names for this technology—DNA microarrays, DNA arrays, DNA chips, gene chips, others.

Microarrays exploit the preferential binding of complementary nucleic acid sequences. A microarray is typically a glass slide, on to which DNA molecules are attached at fixed locations (spots or features). There may be tens of thousands of spots on an array, each containing a huge number of identical DNA molecules (or fragments of identical molecules), of lengths from twenty to hundreds of nucleotides. The spots on a microarray are either printed on the microarrays by a robot, or synthesized by photo-lithography (similar to computer chip productions) or by ink-jet printing. There are commercially available microarrays, however many labs produce their own microarrays.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not deleteriously changed by the presence of more than that which is recited.

Other definitions may appear throughout this disclosure in the appropriate context.

Functional Analysis of Human Hematopoietic Stem Cell Gene Expression Using Zebrafish

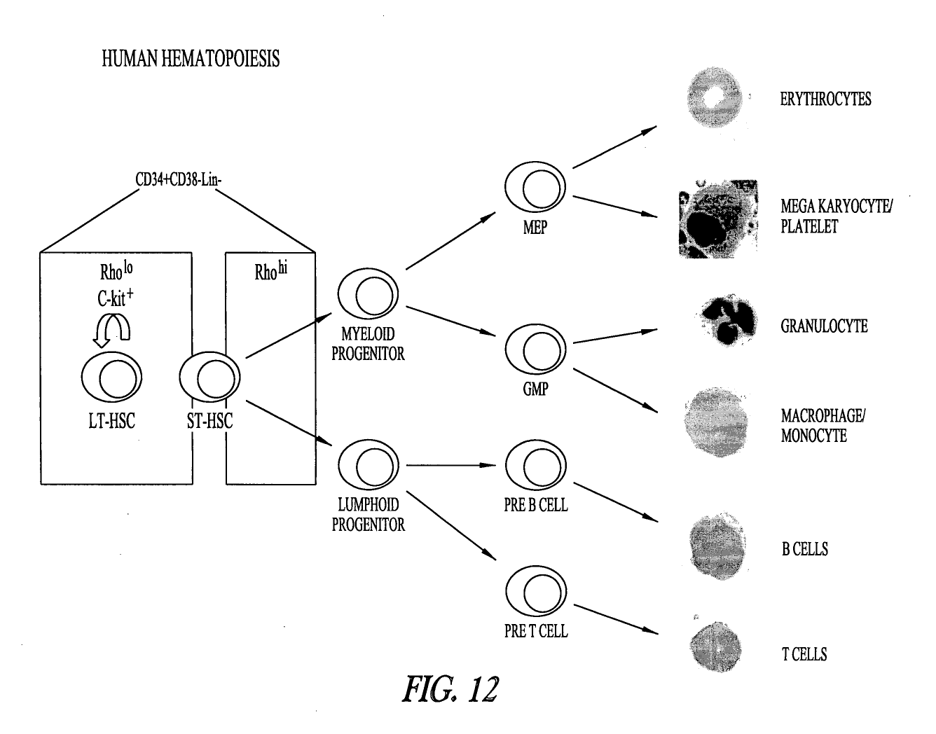

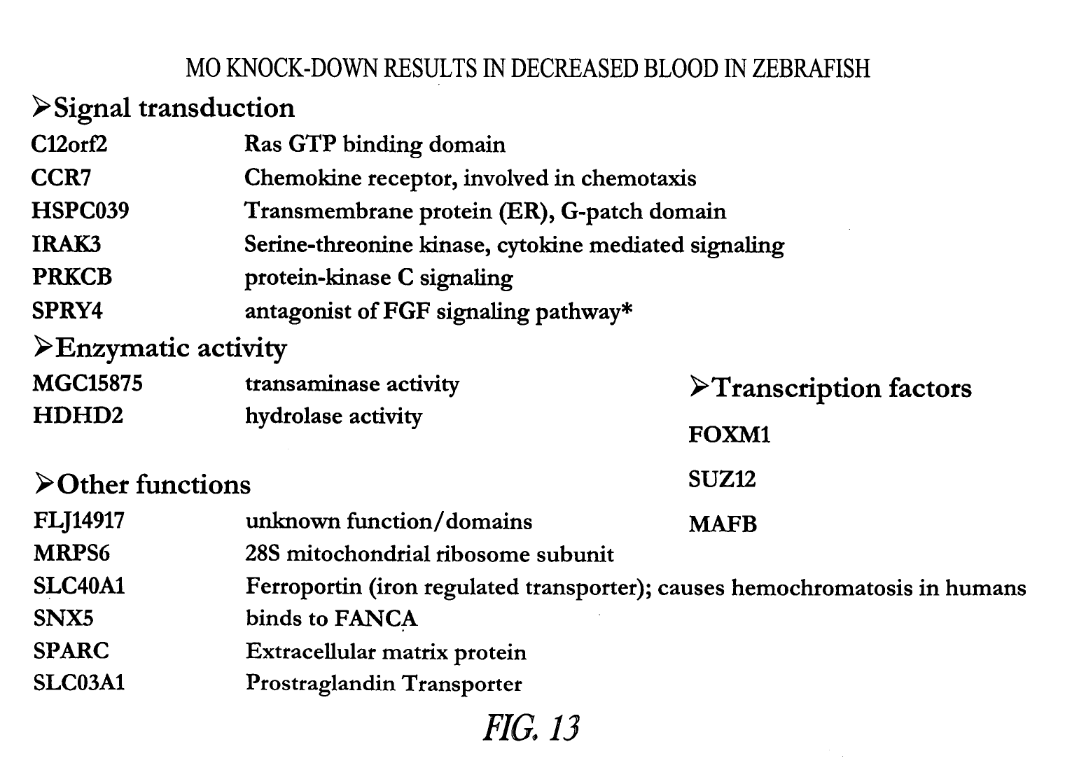

The current understanding of the expressed gene profile of HSCs comes primarily from murine HSCs that can be purified to near homogeneity [10,11,12,13,14]. The difficulty in purifying human HSCs to similar degrees of homogeneity makes study of the transcriptome of human HSCs more difficult. Human HSCs and HPCs are CD34 positive, while cells that engraft in severe combined immune deficiency (SCID) mice are enriched in the CD34⁺Lineage(Lin)⁻ CD38⁻ fraction [15]. As fewer than 1/500 CD34⁺Lin⁻CD38⁻ cells can repopulate SCID mice [15], the expressed gene profile of CD34⁺Lin⁻CD38⁻ cells is likely only partially enriched for HSC-specific genes [12,16]. Previously it was demonstrated that the Rhodamine (Rho) 123⁻ and c-kit⁺ subpopulation of CD34⁺Lin⁻CD38⁻ cells (Rho^{lo}) are highly enriched for primitive HPCs with myeloid-lymphoid-initiating cell (ML-IC) capacity relative to CD34⁺CD38⁻CD33⁻Rho^{hi} (Rho^{hi} ) cells [17; FIG. 12]. Thus, such selection separates CD34⁺Lin⁻CD38⁻ cells into HSC-enriched and HSC-depleted populations.

Comparison of the transcriptome of Rho^{lo} and Rho^{hi} cells from umbilical cord blood (UCB) and bone marrow (BM) identified conserved genes and gene pathways that define the human HSC. Because of the inherent limitations of using gene expression data to infer biological gene function, the hematopoietic role of these genes in a high-throughput in vivo functional genomics screen in the zebrafish was assessed. Using this strategy a series of genes that represent novel regulators of human HSC fate decisions was identified. Further, this work represents the first example of a functional genetic screening strategy that is a large step toward obtaining biologically relevant functional data from global gene profiling studies.

To identify candidate regulators of HSC fate decisions, the transcriptome of human umbilical cord blood and bone marrow CD34⁺CD33⁻CD38⁻Rho^{lo}c-kit⁺ cells, enriched for hematopoietic stem/progenitor cells with CD34⁺CD33⁻CD38⁻Rho^{hi} cells, enriched in committed progenitors, were compared. 277 differentially expressed transcripts conserved in these ontogenically distinct cell sources were identified. A morpholino antisense oligonucleotide (MO)-based functional screen in zebrafish was performed to determine the hematopoietic function of 61 genes that had no previously known function in HSC biology and for which a likely zebrafish ortholog could be identified.

MO knockdown of 14/61 (23%) of the differentially expressed transcripts resulted in hematopoietic defects in developing zebrafish embryos, as demonstrated by altered levels of circulating blood cells at 30 and 48 hours post fertilization and subsequently confirmed by quantitative RT-PCR for erythroid-specific hbae1 and/or myeloid-specific lcp1 transcripts. Recapitulating the knockdown phenotype using a second MO of independent sequence, absence of the phenotype using a mismatched MO sequence and rescue of the phenotype by cDNA-based overexpression of the targeted transcript for zebrafish spry4 confirmed the specificity of MO targeting in this system. Further characterization of the spry4-deficient zebrafish embryos demonstrated that hematopoietic defects were not due to more wide-spread defects in the mesodermal development, and therefore represented primary defects in HSC specification, proliferation and/or differentiation. Overall, this high-throughput screen for the functional validation of differentially expressed genes using a zebrafish model of hematopoiesis represents a major step toward obtaining meaningful information from global gene profiling of HSC.

The following example is put forth so as to provide those of ordinary skill in the art with a complete disclosure and description of how to make and use the invention, and are not intended to limit the scope of what the inventors regard as their invention.

### EXAMPLE

Materials and Methods

Isolation of Rho^{lo} and Rho^{hi} Cell Populations from UCB and BM.

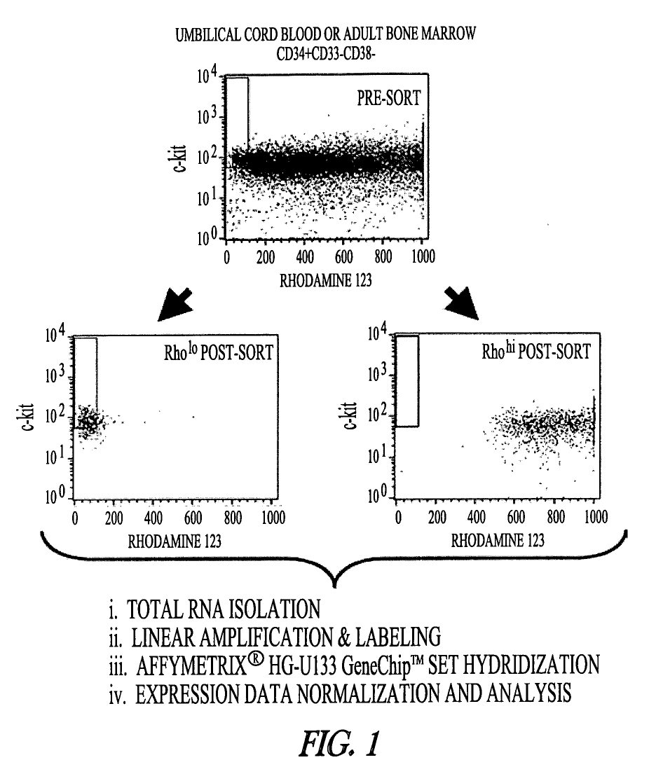

Human UCB from full-term delivered infants and BM from healthy donors were obtained after informed consent in accordance with guidelines approved by the University of Minnesota Committee on the Use of Human Subjects in Research. Each biologically distinct replicate was comprised of one to four donors for UCB and individual donors for BM samples. CD34⁺CD38⁻CD33⁻Rho^{lo}c-kit⁺ and CD34⁺CD38⁻CD33⁻Rho^{hi} fractions were selected by sequential Ficol-Hypaque separation, MACS column depletion and fluorescence activated cell sorting as previously described [ 17]. Post-sort analysis demonstrated that sorted populations contained fewer than 1-2% contaminating cells from the opposing population (FIG. 1).

Determination of ML-IC Frequencies.

ML-IC frequencies for UCB samples (n=3) were determined as previously described [17]. An ML-IC was defined as a single cell that gave rise to at least one LTC-IC and one NK-IC. Results are presented as ML-IC frequency±standard deviation of the mean.

Processing of RNA Samples and Oligonucleotide Microarray Analysis.

Total cellular RNA was isolated from UCB (n=5) and BM (n=4) Rho^{lo} and Rho^{hi} cells using the PicoPure RNA Isolation Kit (Arcturus, Mountain View, Calif., United States) per the manufacturer's instructions. Seven to ten thousand Rho^{lo} and Rho^{hi} cells were sorted directly into 100 μL Extraction Buffer (XB) provided with the PicoPure RNA Isolation Kit (Arctutus) prior to RNA isolation. Labeled complimentary-RNA (cRNA) was generated by one round of IVT-based, linear amplification using the RiboAmp OA RNA Amplification Kit (Arcturus) followed by labeling with the Enzo Bioarray™ HighYield™ RNA Transcript Labeling Kit (Enzo Life Sciences, Farmingdale, N.Y., United States) according to the manufacturer's instructions. Samples were hybridized to Affymetrix™ HG-U133 A & B chips (Affymetrix™ Inc., Santa Clara, Calif., United States), washed and scanned at the University of Minnesota Affymetrix™ Microarray Core Facility as described in the Affymetrix™ GeneChip® Expression Analysis Technical Manual.

Oligonucleotide Microarray Data Analysis.

Affymetrix® HG-U133 GeneChips™ were processed using Genedata Refiner software (GeneData, San Francisco, Calif., United States) to assess overall quality. Feature intensities for each chip were condensed into a single intensity value per gene using the Affymetrix Statistical Algorithm (MAS 5.0) with Tau=0.015, Alpha1=0.04, Alpha2 0.06 and a scaling factor of 500. Expression data was analyzed using GeneData's Expressionist and Microsoft Excel (Microsoft, Redmond, Wash., United States). Differential gene expression for comparison of Rho^{lo} versus Rho^{hi} cells was defined by using a paired Student's t-test with a threshold of p<0.05, and a paired fold change was used to rank gene lists. Differentially expressed genes were classified according to their respective gene pathways and gene ontologies when available by using the web-based Affymetrix™ NetAffx analysis tool (http://www.affymetrix.com) and the National Institutes of Allergy and Infectious Disease (NIAID) Database for Annotation Visualization and Integrated Discovery (DAVID) analysis tool (http://alps1.niaid.nih.gov/david).

Microarray Q-RT-PCR Confirmation.

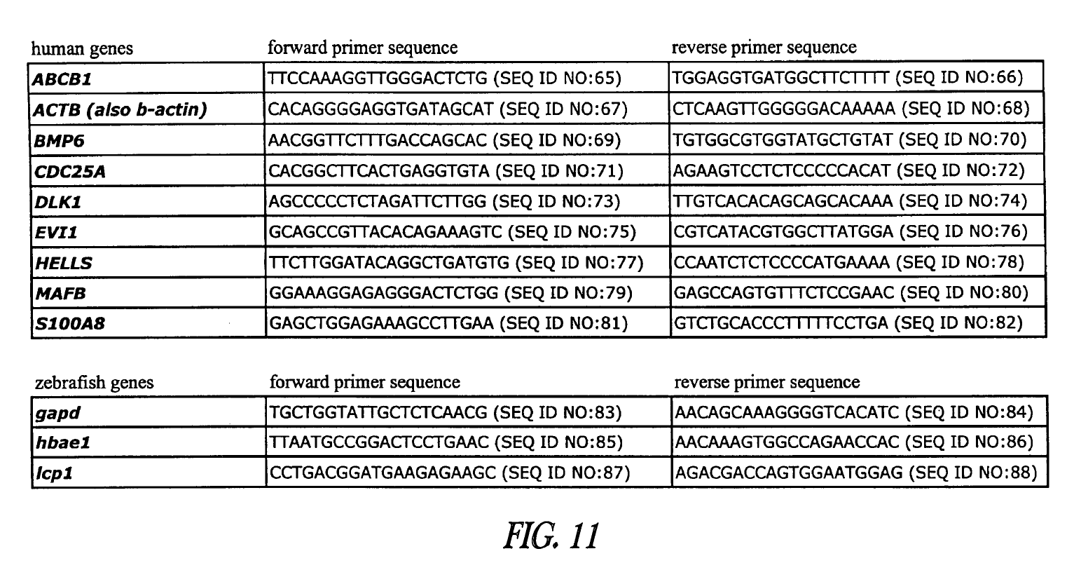

Labeled cRNA was reverse transcribed to generate cDNA using SuperScript™ II Reverse Transcriptase (Invitrogen, Carlsbad, Calif., United States) according to the manufacturer's instructions. Quantitative, real-time PCR (Q-RT-PCR) was performed using the ABI Prism® 7000 Sequence Detection System (Applied Biosystems, Foster City, Calif., United States). Briefly, 3 ng of cDNA was amplified by 40 cycles of a two-step PCR reaction (95° C. for 15 sec. denaturation and 60° C. for 1 min. annealing/elongation) containing 100 nM gene-specific primers (FIG. 11) and the SYBR® Green PCR Master Mix (Applied Biosystems). Gene expression was normalized using human ACTB (also β-actin) expression levels. Results are presented as percent expression relative to uninjected clutchmate control embryos±standard deviation of the mean.

High-throughput Loss-of-function Genetic screen in Zebrafish.

The likely zebrafish orthologs of differentially expressed human genes were identified using Ensembl's gene homology prediction program (http://www.ensemble.org, build Zv3) in combination with comparison of the human protein sequence to The Institute for Genomics Research zebrafish EST database (http://www.tigr.org, release 13-15). The criteria for a likely ortholog was ≧40% amino acid identity over the entire length of the protein or ≧50% if the fish protein sequence was only partial.

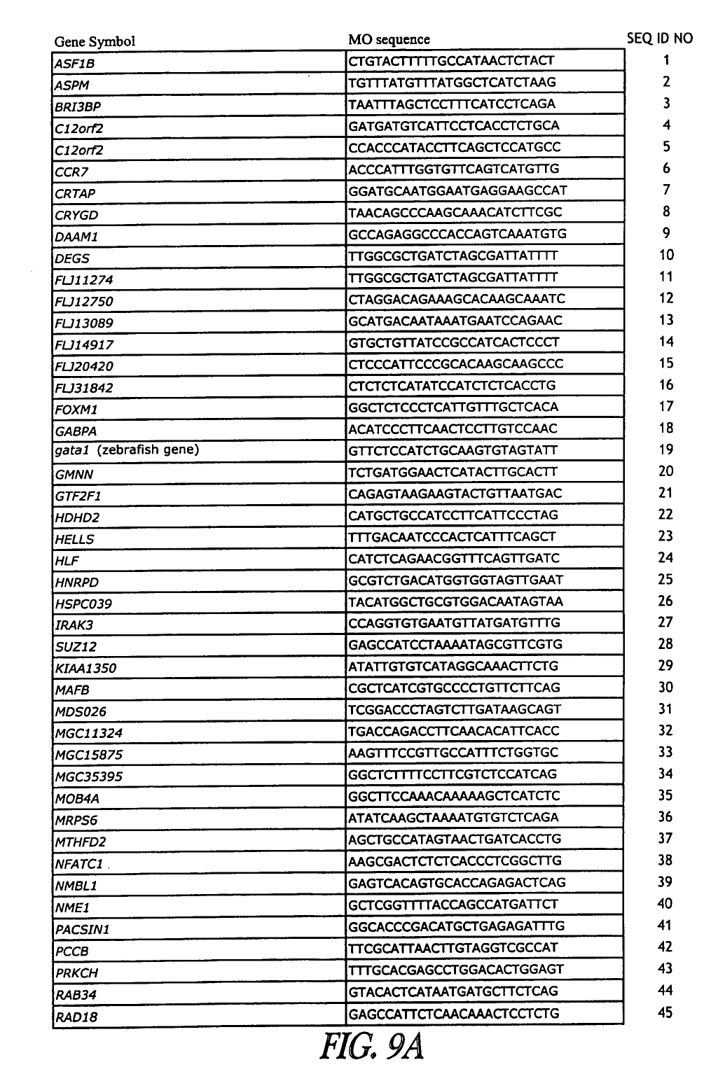

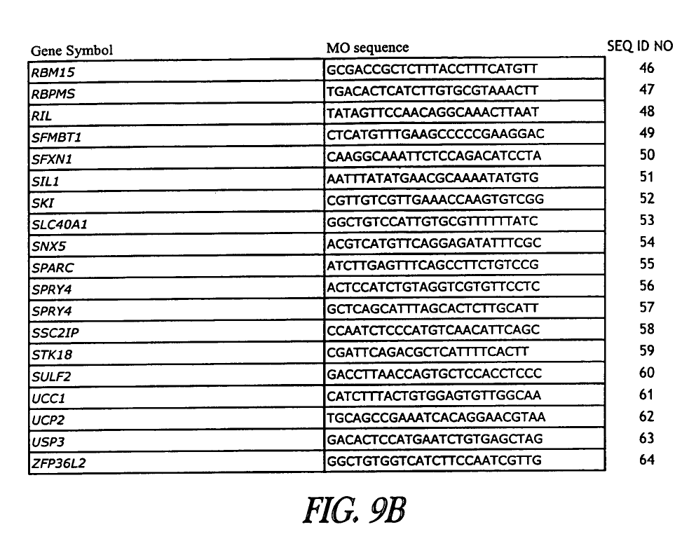

Morpholino oligos are short chains of Morpholino subunits comprised of a nucleic acid base, a morpholine ring and a non-ionic phosphorodiamidate intersubunit linkage. Morpholinos are believed to act via a steric block mechanism (RNAse H-independent). Morpholino antisense oligonucleotide (MO) (Gene Tools, Philomath, Oreg., United States) sequences were designed complimentary to the region of translational initiation of the zebrafish orthologs in order to inhibit protein translation (FIG. 9) and injected into one- to two-cell zebrafish embryos as previously described [36]. MOs were injected at 1.5, 3, 4.5 and 6 ng into 50-70 embryos derived from mating transgenic zebrafish containing a fluorescent DsRed reporter cassette driven by the gata1 promoter (gata1:DsRed Tg) (provided by Dr. Leonard I. Zon, Harvard University, Boston, Mass.) or a fluorescent eGFP reporter cassette driven by the fli 1 promoter (fli1:eGFP Tg). MO-targeted (morphant) zebrafish were evaluated for defects in hematopoietic and vascular development compared to uninjected controls from the same embryo clutch at 30 and 48 hours post fertilization (hpf) using fluorescence microscopy for visualization of DsRed⁺ blood cells and eGFP⁺ vasculature.

Whole Zebrafish Q-RT-PCR Confirmation.

Total RNA was isolated from five MO-injected zebrafish embryos at 48 hpf with hematopoietic defects, determined based on gata1:DsRed⁺ hematopoietic cell production, or five uninjected clutchmate controls using the RNeasy Mini Kit (QIAGEN, Valencia, Calif., United States) according to the manufacturer's instructions. Total RNA was incubated with DNaseI (Invitrogen) to digest contaminating genomic DNA, and reverse transcribed to generate cDNA using SuperScript# II Reverse Transcriptase (Invitrogen) according to the manufacturer's protocol. Quantitative, real-time PCR was performed using the ABI Prism® 7000 Sequence Detection System (Applied Biosystems). Briefly, 1/20 of the total cDNA from five zebrafish embryos was amplified by 40 cycles of a two-step PCR reaction (95° C. for 15 sec. denaturation and 60° C. for 1 min. annealing/elongation) containing 100 nM gene-specific primers (FIG. 11) and the SYBR® Green PCR Master Mix (Applied Biosystems). Gene expression was normalized using zebrafish gapd expression levels. Results are presented as percent expression relative to un-injected clutchmate control embryos±standard deviation of the mean.

Whole-mount in situ Hybridization.

Scl, myod, flk1, gata1, and cmyb riboprobes were generated and whole- mount in situ hybridization of zebrafish embryos was conducted as previously described [41].

Overexpression of Human and Zebrafish Sprouty Gene Family Members.

The XhoI and Kpnl fragment of the SPRY1 open reading frame (ORF) (Open Biosystems, Huntsville, Ala., United States) was cloned into pENTRIA (Invitrogen) and subsequently transferred into a modified pFRM2.1 zebrafish expression vector using the Gateway cloning system™ (Invitrogen) to create the pFRM2.1₋SPRY1 vector. pFRM2.1_SPRY1 was co-injected with pFRM2.1_eGFP at a 5:1 ratio into the yolk/cell interface of one cell gata1:DsRed Tg zebrafish embryos as described for MO injections. Defects in hematopoietic development of eGFP⁺ embryos were analyzed by comparison to embryos from the same clutch injected with pFRM2.1_eGFP alone using fluorescence microscopy to visualize DsRed⁺ blood cells.

Accession Numbers

The National Center for Biotechnology Information (http://www.ncbi.nlm.nih.-gov/): Entrez Gene accession numbers for the following genes are: ABCB1(5243), ARMCX2 (9823), BMP4 (652), C12or f2 (11228), CCR7 (1236), Ccr7 (12775), CDKN1A (1026), chd (30161), cmyb (30519), CRYGD (1421), EVI1 (2122), EZH2 (2146), FLJ14917 (84947), flk1 (also known as kdr) (58106), FOXM1 (2305), gata1 (30481), GATA2 (2624), gata2 (30480), hbae1 (30597), HDHD2 (84064), HELLS (3070), HLF (3131), HMGA2 (8091), Hoxb4 (15412), HOXB4 (3214), HSPC039 (51124), IRAK3 (11213), Irak3 (73914), KIAA1102 (22998), KLF5 (688), lcp1 (30583), LEF1 (51176), LMO2 (4005), lmo2 (30332), MAFB (9935), MGC15875 (85007), MRPS6 (64968), myod (30513), NOTCH2 (4853), PIM1 (5292), PRKCH (5583), Prkch (18755), RBPMS (11030), scl(also known as tal1) (30766), SHH (6469), SLC40A1 (30061), SLC03A1 (28232), SNX5 (27131), SPARC (6678), Sparc (20692), spry4 (114437), SPRY1 (10252), SSBP2 (23635), SUZ12 (23512), ZFHX1B (9839), ZNF165 (7718), and ZNF331 (55422). The microarray data have been deposited in the GEO database (http://www.ncbi.nlm.nih.gov/geo/), and have been assigned the accession number GSE2666. The genes and microarray data are incorporated herein by referenced.

Results and Discussion

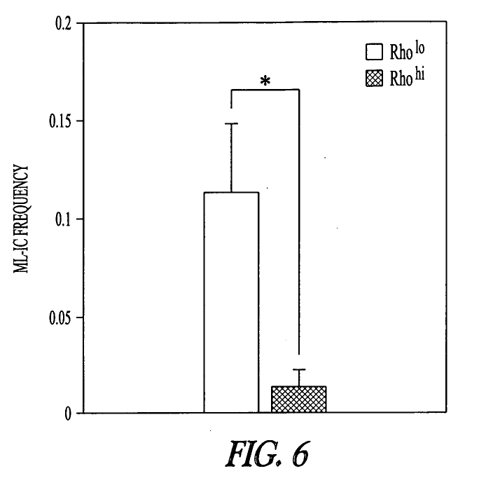

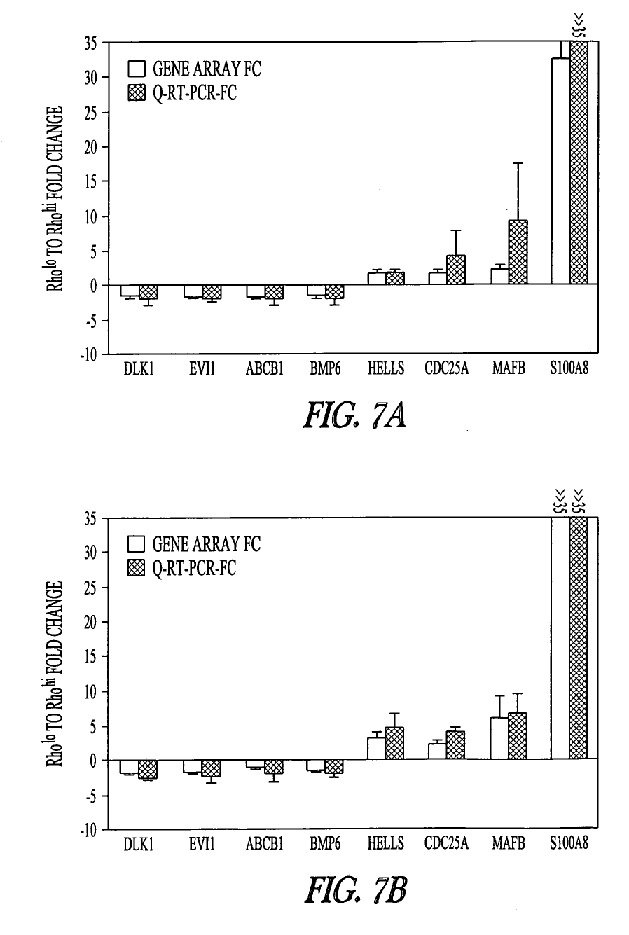

Myeloid-Lymphoid initiating Cells (ML-ICs) are highly enriched in Rho^{lo} compared to Rho^{hi} cells. In the past, the study of human HSCs has been limited since the CD34⁺Lin⁻CD38⁻ fraction of hematopoietic cells, commonly used as an HSC enriched population, contains fewer than 0.2% SCID-repopulating cells [15], suggesting considerable heterogeneity. ML-ICs, single hematopoietic cells that can generate several daughter cells that are capable of re-initiating long-term myeloid and long-term lymphoid cultures, were highly enriched by selecting the Rho^{lo} fraction of CD34⁺Lin⁻CD38⁻ cells. While the Rho^{lo} population still only contains 15-25% ML-ICs and therefore remains heterogeneous, the enrichment factor is about 5- to 10-fold greater than CD34⁺Lin⁻CD38⁻ cells [17]. The ML-IC frequency was ≧10-fold higher in UCB Rho^{lo} compared to Rho^{hi} cells (FIG. 6).

Genes differentially expressed between Rho^{lo} and Rho^{hi} cells from both UCB and BM. Comparing genes differentially expressed between Rho^{lo} and Rho^{hi} cells from ontogenically distinct sources identified conserved genes and gene pathways that govern self-renewal and differentiation of human HSCs. The experimental design used is illustrated in FIG. 1. Differentially expressed probe sets were defined as those with a p-value<0.05 using a paired Student's t-test. By taking into account the variability present in primary cell populations, this provides a more accurate analysis of differential gene expression compared with the commonly used fold change cutoff.

2,707 and 4,667 probe sets differentially expressed between Rho^{lo} and Rho^{hi} cells from UCB and BM were identified (as presented in U.S. Ser. No. 60/690,089, which is herein incorporated by reference for the description of the differentially expressed probe sets, including confirmation Q-RT-PCR and the 277 unique transcripts). The fidelity of the microarray results was confirmed using quantitative RT-PCR (Q-RT-PCR). Further analysis was focused on 277 unique transcripts, represented by 304 probe sets that were differentially expressed between Rho^{lo} and Rho^{hi} cells from both UCB and BM with a fold change >1.5 in either UCB or BM.

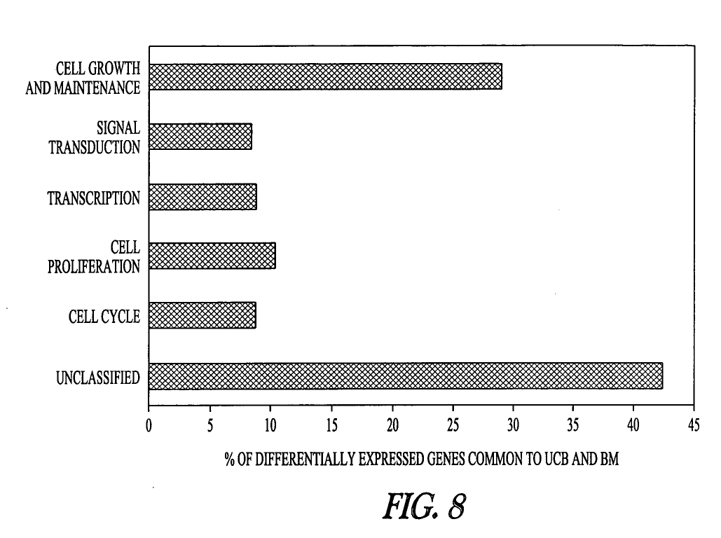

Among the conserved genes enriched in Rho^{lo} cells, many have been implicated in early hematopoiesis, including CDKN1A (GeneID: 1026), a cell cycle regulator for maintenance of murine HSCs [18], and ABCB1 (GeneID: 5243), the ABC-transporter family member responsible for the Rho^{lo} phenotype [17]. Several transcription factors (TFs) known to play a role in early hematopoiesis or leukemogenesis were also identified, including HLF (GeneID: 3131), involved in leukemogenic chromosomal translocations [19] and EVI1 (GeneID: 2122), a TF associated with myeloid leukemias [20]. Other TFs without a known role in hematopoiesis were also more highly expressed in Rho^{lo} cells, including HMGA2 (GenefD: 8091), a high mobility group gene; and the zinc finger TFs ZNF165 (GeneID: 7718), ZNF331 (GeneID: 55422) and KLF5 (GeneID: 688). All Rho^{lo}-enriched genes are listed in U.S. Ser. No. 60/690,089, which is herein incorporated by reference for the list of Rho^{lo}-enriched genes. As demonstrated in previous HSC gene profiling studies [12,13,14], >40% of genes enriched in Rho^{lo} cells lack a functional annotation, are hypothetical proteins or are expressed sequence tags (ESTs), and thus represent currently uncharacterized regulators of HSC fate decisions (FIG. 8).

Some genes with well-established roles in HSC self-renewal and early differentiation are not present in the Rho^{lo} enriched gene list. However, most of these were differentially expressed in both datasets, but differences did not reach statistical significance. For instance, LMO2 (GeneID: 4005) [21] and GATA2 (GeneID: 2624) [22], known to be involved in HSC development and self-renewal, were expressed significantly higher in BM Rho^{lo} than Rho^{hi} cells. Although similar trends were seen in UCB Rho^{lo} cells, these differences were not statistically significant. Conversely, HOXB4 (GeneID: 3214) [4] expression was significantly higher in UCB Rho^{lo} than Rho^{hi} cells, but this difference was not statistically significant in BM. Although the stringent criteria for differential expression likely contribute to the omission of some genes that might be differentially expressed, another explanation might be that expression of these genes is maintained when cells differentiate from a Rho^{lo} to a Rho^{hi} stage. The latter is consistent with most known HSC-associated genes expressed at levels that are much higher than the normalized average microarray expression levels in Rho^{lo} and Rho^{hi} cells from both UCB and BM.

Conserved genes enriched in the Rho^{hi} cells included LEF1, an effector of Wnt signaling expressed in pre-B and T cells [23], and NOTCH2 (GeneID: 4853), involved in hematopoietic differentiation cell fate decision [24]. Several TFs known to play a role in hematopoietic cell differentiation were more highly expressed in Rho^{hi} compared to Rho^{lo} cells, including HELLS (GeneID: 3070) and MAFB (GeneID: 9935) [25,26]. Additional TFs with no known role in hematopoietic development were also enriched in Rho^{hi} cells, such as the zinc finger homeobox gene, ZFHX1B (GeneID: 9839), and the polycomb genes, EZH2 (GeneID: 2146) and SUZ12 (GeneID: 23512), the later plays a role in germ cell development [27]. Globin (Hb) gene family members were also more highly expressed in UCB and BM Rho^{hi} than Rho^{lo} cells. Consistent with the ontogenic expression patterns of fetal versus adult Hb genes, Hbγ genes were more highly expressed in perinatal UCB Rho^{hi} cells, while Hbβ genes were more highly expressed in adult BM Rho^{hi} cells. Additional genes enriched in Rho^{hi} cells are listed in U.S. Ser. No. 60/690,089, which is incorporated by reference for the list of genes enriched in Rho^{hi} cells.

Because of functional redundancy amongst gene families, the data for common differentially expressed gene family members were examined. The Id family of transcriptional repressors [28] was enriched in the Rho^{lo} fraction, but was represented by different family members in UCB (ID4) and BM (ID1, ID2 and ID3). Similarly, various H1 and H2 histone genes were enriched in the Rho^{lo} fraction in both datasets, but were represented by distinct family members.

It was also evaluated whether common differentially expressed genes were concentrated on specific chromosomes. It was found that genes were not only concentrated on certain chromosomes, but at specific g-band addresses. Of the genes enriched in Rho^{lo} cells, 9% reside at 6p21, a region involved in recurrent chromosomal translocations in myeloid [29] and lymphoid [30] leukemias, and home to the PIM1 oncogene (GeneID: 5292) [31]. Six members of the H2B and one member of the H1 histone family as well as CDKN1A, more highly expressed in Rho^{lo} than Rho^{hi} cells, reside at 6p21. The remaining Rho^{lo}-enriched genes at 6p21 consist of six class II major histocompatibility complex (MHC) family members and a putative testis specific zinc finger TF, ZNF165. H1 and H2 histone gene family members [11,13], class II MHC antigens [12,13] and CDKN1A [13] were also found amongst the genes identified in studies characterizing the transcriptome of murine HSC. The differential expression of such a large number of genes located at this chromosomal address, suggests that like CDKN1A, other genes located at 6p21 with as yet unknown hematopoietic function can a role in HSC proliferation or differentiation.

The genes expressed more highly in Rho^{lo} versus Rho^{hi} cells were compared with published gene expression data. Comparison with the study by Ivanova et al. [12] that compared human CD34⁺Lin⁻CD38⁻ with CD34⁺Lin⁻CD38⁺ cells, yielded only seven genes in common: ARMCX2 (GeneID: 9823), CRYGD (GeneID: 1421), HLF, K1AA1102 (GeneID: 22998), RBPMS (GeneID: 11030), SLCO3A1 (GeneID: 28232) and SSBP2 (GeneID: 23635). The lack of overlap is that surprising, as Rho^{lo} and Rho^{hi} cells are subpopulations of the CD34⁺Lin⁻CD38⁻ population used by Ivanova et al. Comparison of genes expressed more highly in Rho^{lo} versus Rho^{hi} cells with genes expressed more highly in murine side population (SP)/KLS/CD34⁻ compared to total BM cells published by Ramalho-Santos et al. [13] identified 16 likely orthologs and 38 common gene family members (this comparison is presented in U.S. Ser. No. 60/690,089 , which is herein incorporated by reference), suggesting that HSC specific genes are conserved across species.

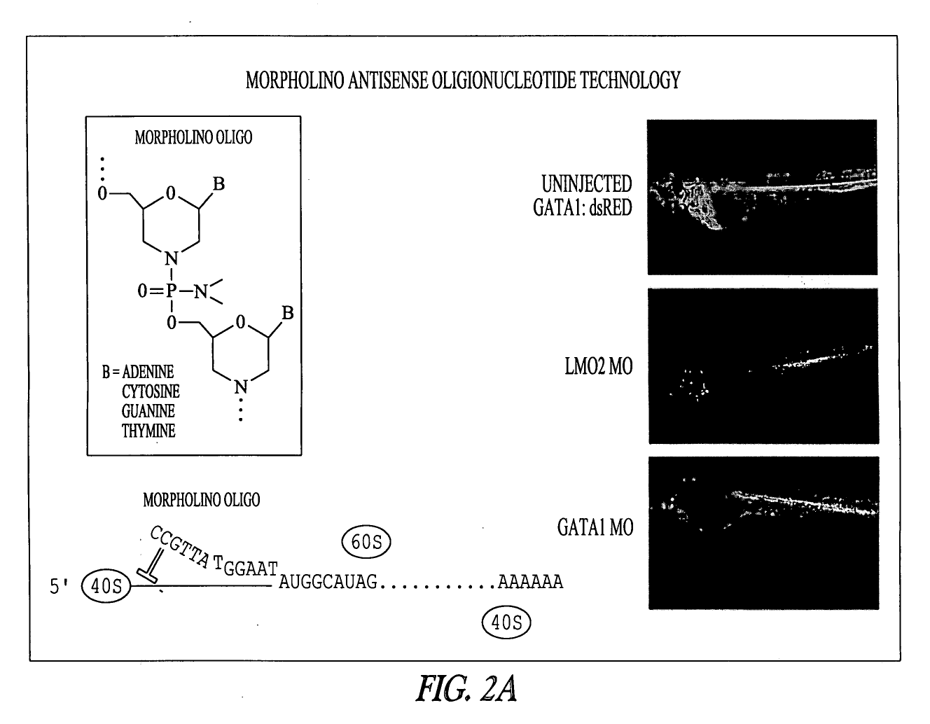

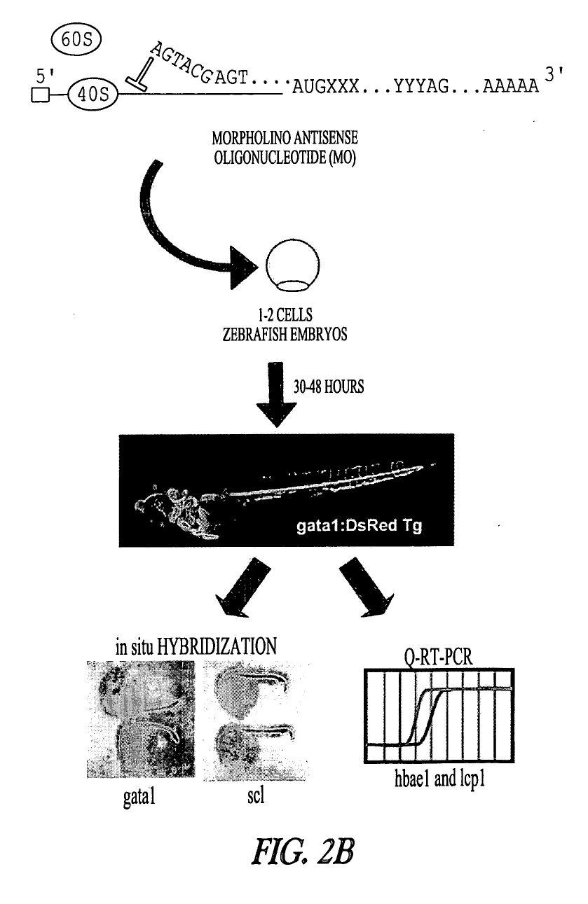

In vivo fimctional genomics screen in zebrafish. Because gene-profiling per se does not prove functional importance, an in vivo functional genomics screen in zebrafish was developed (FIG. 2). The zebrafish, Danio rerio, is an ideal organism for high-throughput genetic screens [32] as organogenesis is highly conserved from zebrafish to man [33]. There is abundant evidence that hematopoiesis in zebrafish occurs via a highly conserved genetic program. As in mammals, hematopoiesis in zebrafish occurs via specification of mesoderm to a hemangioblast stage that subsequently commits to either HSC or angioblasts [34], and genes and signals involved in specification (BMP signaling) and commitment (Vascular Endothelial Growth Factor (VEGF) signaling, flkI (also known as kdr; GeneID: 58106), lmo2 (GeneID: 30332), scl (also known as tal1; GeneID: 30766), gata2 (GeneID: 30480), gata1 (GeneID: 30481)) are conserved from fish to man [35]. This high degree of homology in the genetic control of zebrafish and human hematopoietic development makes genetic screens in zebrafish a powerful tool to elucidate the role of genes in hematopoiesis. Additionally, rapid reverse genetic screens can be accomplished using morpholino antisense oligonucleotides (MOs) to knockdown gene expression in the developing zebrafish embryo [36].

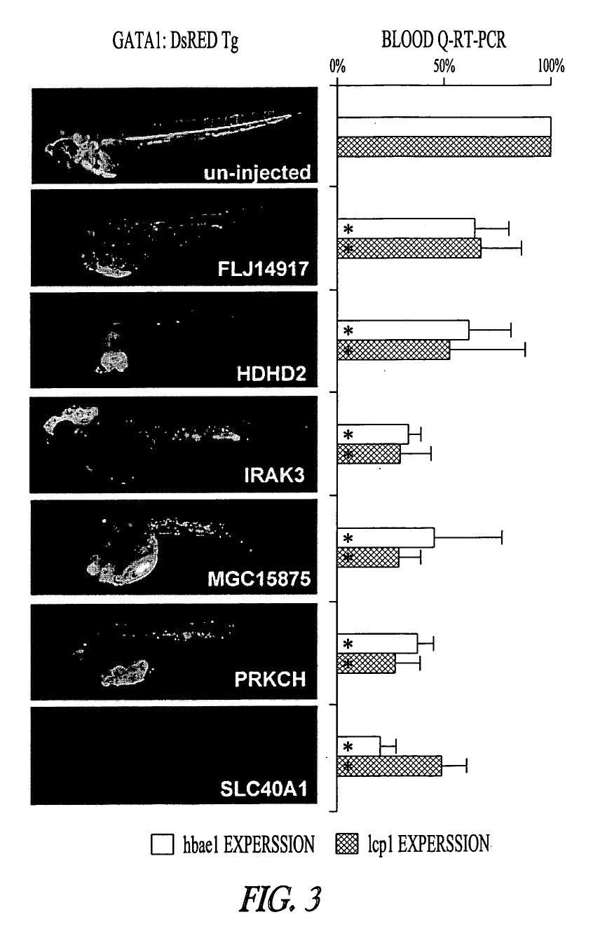

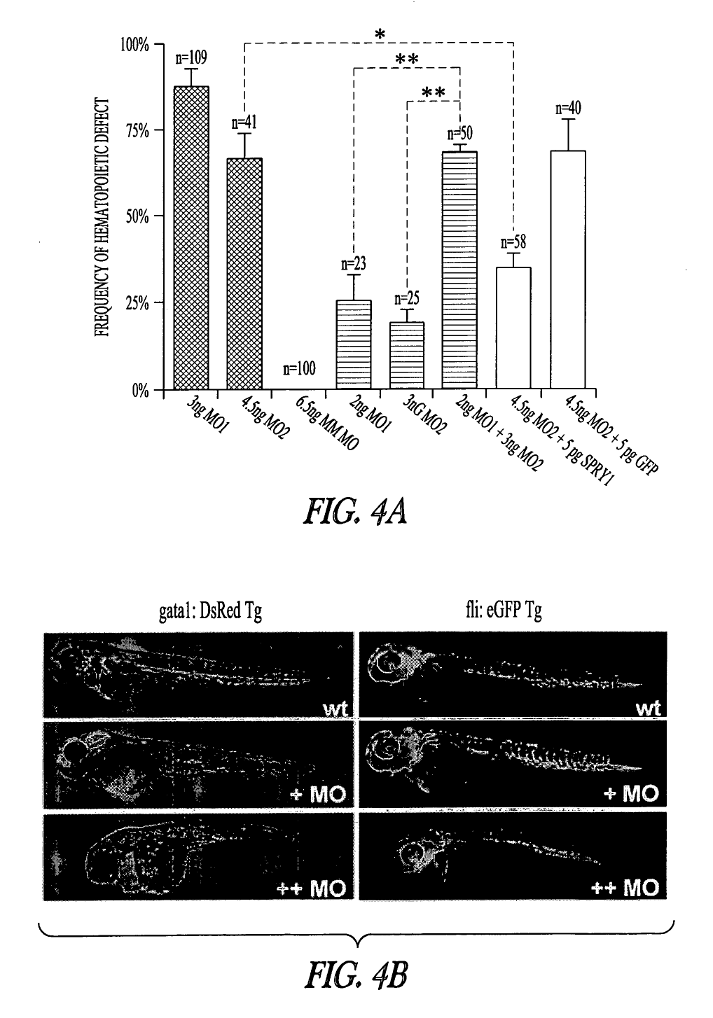

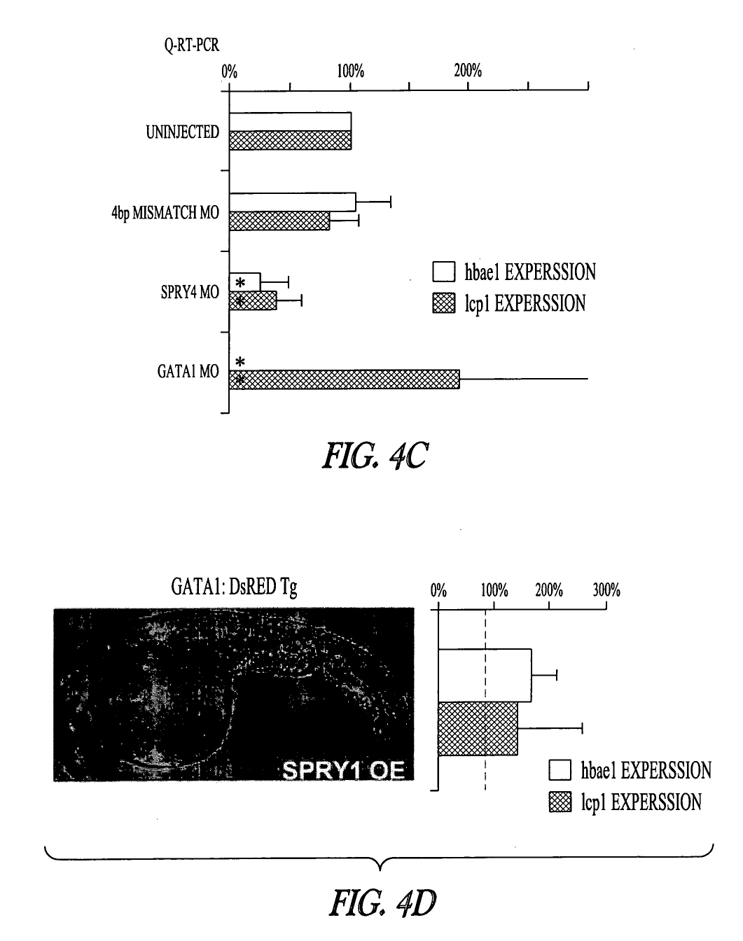

From the 277 unique transcripts that were differentially expressed between Rho^{lo} and Rho^{hi} cells of both UCB and BM, genes with known function in hematopoiesis, MHC genes, histones, and genes that are known to play a role in glucose and protein metabolism and RNA and DNA synthesis were eliminated, resulting in a final list of 158 genes. Of these, a putative zebrafish ortholog for 86 was identified, and MOs were designed against 61 (FIG. 9; 85 genes have been screened (data not shown for all 85)). The 61 MOs were injected in 1- to 2-cell zebrafish embryos and assayed for effects on blood development. Initial dosing studies identified 16/61 MOs that reduce blood cell production without confounding toxicities (Table 1). The 16 MOs induced a blood defect in >70% of embryos in two or more independent injections. Blood defects identified by gata1:DsRed transgenic (Tg) fluorescence microscopy were confirmed by Q-RT-PCR of the erythroid-specific hbae1 (GeneID: 30597) and myeloid-specific lcp1 (GeneID: 30583) transcripts in MO-targeted embryos compared to uninjected controls in ≧3 independent experiments of n=5 embryos per experiment. A >2-fold reduction in both erythroid and myeloid gene expression levels were seen for 5 of 7 MOs analyzed (Table 1, FIG. 3 and FIG. 4). Thus, the addition of Q-RT-PCR to the screening process provides an independent confirmation and quantitation of the observed phenotypes, thereby limiting the false-positive rate, while maintaining the high-throughput nature of the screen.

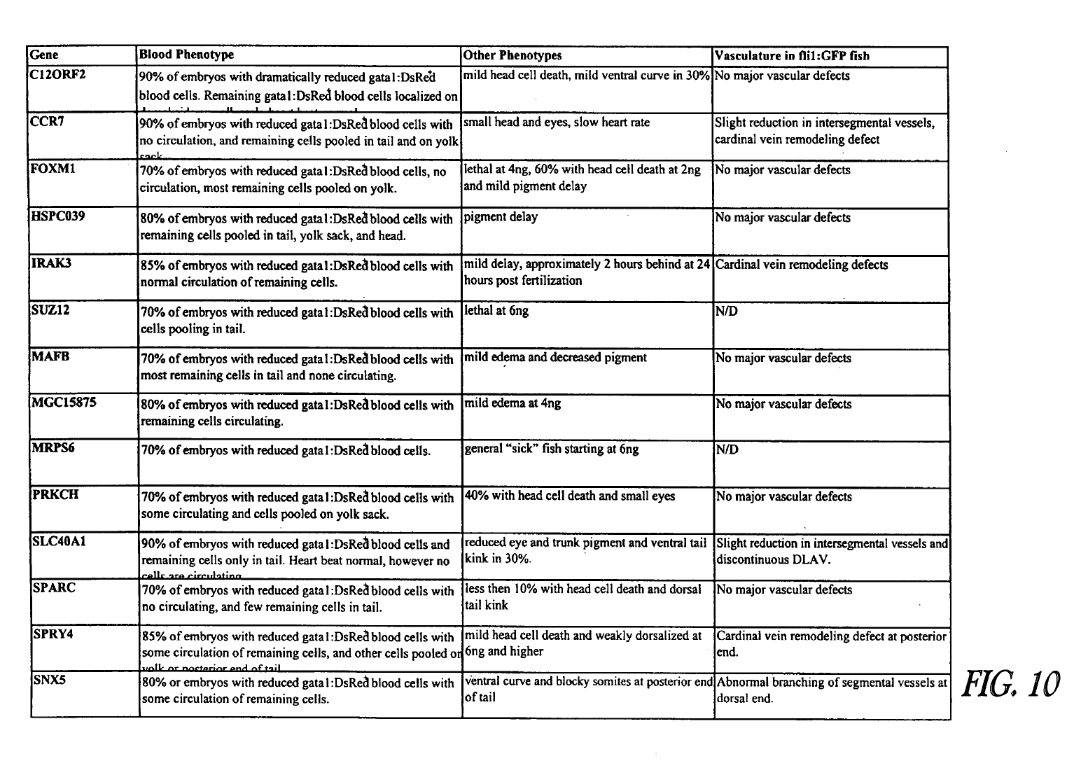

Additionally, the observed reduction of both erythroid and myeloid gene expression following knockdown of candidate genes is consistent with their presumed roles in HSC fate decisions prior to specification of the common myeloid progenitor. The validity of the Q-RT-PCR analysis was corroborated by analysis of hbae1 and lcp1 transcript levels in gata1 MO targeted embryos, in which there was a virtually complete loss of hbae1 expression and an almost 2-fold increase in myeloid-specific lcp1 transcripts (FIG. 4) consistent with the published expression patterns for these genes following loss of gata1 expression [37]. Analysis of vascular development by injecting MOs into fli1:eGFP (enhanced green fluorescent protein) Tg embryos which express eGFP in endothelial cells, demonstrated no major abnormalities in vascular morphogenesis or remodeling that precludes the circulation of the remaining blood cells, indicating that the hematopoietic defect is not secondary to a vascular phenotype (FIG. 4 and FIG. 10).

TABLE 1
Genes differentially expressed between human Rho^{lo} and Rho^{hi} cell populations that have a
functional role in zebrafish hematopoietic development (ATG, start codon; 5′ UTR, 5′ un-translated
region; Q-RT-PCR, quantitative RT-PCR confirmation; N/D, not done).

| Human Gene | Region Targeted | Dose | | Hematopoietic Phenotype | Expression | Q-RT-PCR |
|---|---|---|---|---|---|---|
| C12ORF2 | 5′ UTR | 4.5 | ng | 90% with blood defect | Rho^{lo} | N/D |
| | ATG | 4.5 | ng | 95% with blood defect | | |
| | combination | 3 | ng each | 95% with blood defect | | |
| CCR7 | ATG | 2 | ng | 90% with blood defect | Rho^{hi} | N/D |
| FLJ14917 | ATG | 7 | ng | 80% with blood defect | Rho^{lo} | not confirmed |
| FOXM1 | ATG | 2 | ng | 70% with blood defect | Rho^{hi} | N/D |
| HDHD2 | ATG | 3 | ng | 75% with blood defect | Rho^{hi} | not confirmed |
| HSPC039 | 5′ UTR | 3 | ng | 80% with blood defect | Rho^{hi} | N/D |
| IRAK3 | 5′ UTR | 2 | ng | 70% with blood defect | Rho^{lo} | confirmed |
| SUZ12 | ATG | 3 | ng | 70% with blood defect | Rho^{hi} | N/D |
| MAFB | ATG | 4 | ng | 70% with blood defect | Rho^{hi} | N/D |
| MGC15875 | ATG | 4 | ng | 80% with blood defect | Rho^{lo} | confirmed |
| MRPS6 | 5′ UTR | 4.5 | ng | 70% with blood defect | Rho^{hi} | N/D |
| PRKCB | 5′ UTR | 4.5 | ng | 70% with blood defect | Rho^{lo} | confirmed |
| SLC40A1 | ATG | 6 | ng | 90% with blood defect | Rho^{hi} | confirmed |
| SLC03A1 | | | | | | |
| SNX5 | ATG | 5 | ng | 80% with blood defect | Rho^{hi} | N/D |
| SPARC | ATG | 7.5 | ng | 70% with blood defect | Rho^{hi} | N/D |
| SPRY1 | ATG | 3 | ng | 80% with blood defect | Rho^{lo} | confirmed |
| | 5′ UTR | 4.5 | ng | 65% with blood defect | | |

| | | |
|---|---|---|
| combination | 2 ng + 3 ng | 70% with blood defect |

The greater than 20% frequency of blood defects seen in the screen compares very favorably with the 0.5-1% frequency of hematopoietic phenotypes seen by ethylnitrosourea (ENU) mutagenesis screens that mutate genes in a near random fashion [38] and the 4% of hematopoietic phenotypes seen in a morpholino-based finctional screen of the zebrafish secretome [39] (S.C. Ekker, unpublished data). The high incidence of blood defects also demonstrates that the candidate genes identified by comparing the transcriptome of Rho^{lo} and Rho^{hi} cells represent genes with important roles in HSC biology. A candidate ortholog in zebrafish for 72/158 of the differentially expressed human genes was not identifiable. This may be because currently only one quarter of the zebrafish genome is high-quality finished sequence. Hence, some genes with important roles in hematopoiesis may have been untested. However, from a sample of 10 genes that lacked a zebrafish match, only one has a likely ortholog in the Fugu or Medaka sequencing projects, and thus incomplete genome coverage provides only a partial explanation. Alternative possibilities include a reduced level of primary sequence conservation between functional orthologs that may have been missed using the comparative genomics criteria presented herein, or that a number of genes are not conserved between fish and man, and thus might be less important for the conserved processes of hematopoietic self-renewal and differentiation. Therefore, the relatively high incidence of blood defects in conserved genes may in part reflect “evolutionary filtering” in the screen. Consistent with this hypothesis, all zebrafish genes whose mutation resulted in a visible embryonic phenotype identified using a retroviral insertion strategy have a likely human ortholog [40].

Although the frequency of blood defects is high, the screen is not as well suited for the identification of knockdown phenotypes that result in increased HSC proliferation and/or differentiation. The inventors and others have successfully shown that dramatically increased hematopoietic development can be modeled in zebrafish, as is the case for knockdown of the BMP-antagonist chordin (GeneID: 30161) and the corresponding dino mutant [41,42]; however, more modest increases in hematopoietic cell production or skewing of lineage differentiation may be undetectable. Although these caveats may lead to underestimation of the true frequency of genes with a role in early hematopoietic development and differentiation, the screening procedure used has proven effective for extracting functional information from a global gene expression profiling dataset in a high-throughput manner.

Of note, viable knockout mice exist for 4/14 genes identified in the functional screen (Sparc (GeneID: 20692), Irak3 (GeneID: 73914), Ccr7 (GeneID: 12775) and Prkch (GeneID: 18755)) [43,44,45,46]. One could argue that if a viable knockout mouse exists, the gene of interest may not be important in hematopoiesis. However, lack of an overt hematopoietic phenotype does not preclude a role of a gene in HSC self-renewal and differentiation, as this may only be detectable under conditions where the hematopoietic system is stressed or in transplantation experiments. For example, HoxB4^{-/-} mice develop normally, and present with only subtle differences in spleen and BM cellularity [47]. However, the proliferative response of HSC in vitro and in vivo is decreased, consistent with the observation that over-expression of HoxB4 supports expansion of competitive repopulating units and SCID-repopulating cells [4,5].

Characterization of Sprouty family members in zebrafish hematopoiesis. To further verify the hematopoietic role of genes identified by gene array, a more extensive evaluation of the zebrafish targeted with a MO against spry4 (GeneID: 114437) (spry4 morphant or spry4^{MO}) was performed. Although human SPRY1 (GeneID: 10252) was differentially expressed, a MO against zebrafish spry4 was used, as it is expressed in the region of the lateral plate mesoderm, the first site of zebrafish hematopoiesis [48], and it was the full-length zebrafish Sprouty gene with the greatest protein homology to human SPRY1. Recently the partial sequence of a potential zebrafish spry1 ortholog was predicted by Ensemble's gene prediction software based on genomic sequence information. However, the single exon that was predicted does not contain an ATG start codon, or a conserved splice donor or acceptor site, and the putative zebrafish spry1 sequence only partially covers the human SPRY1 gene. Moreover, the genomic location of Ensemble's putative zebrafish spry1 is currently not known, and therefore it is not possible to use syntenic relationships to determine the most likely zebrafish ortholog for human SPRY1. At present, there is not sufficient sequence data available to design gain- or loss-of-function experiments for the putative zebrafish spry1, thus precluding an analysis of hematopoietic function in the zebrafish model. Therefore, spry4 is currently the best full-length, MO-targetable candidate ortholog for human SPRY1, and based on the results, at the very least zebrafish spry4 and SPRY1 share a conserved function in hematopoiesis.

To confirm the specificity of MO targeting in the spry4^{MO}, a second spry4 MO of independent sequence and a 4-base mismatched spry4 MO were injected into zebrafish embryos. Injection of the independent spry4 MOs induced a hematopoietic phenotype in >65% of injected embryos, while the 4-base mismatched MO did not induce any phenotypic changes (FIG. 4). Q-RT-PCR for hbae1 and lcp1 transcripts also did not show changes in expression in 4-base mismatched MO embryos. Furthermore, the two independent spry4 MOs acted synergistically when co-injected (FIG. 4). In addition to the blood phenotype, a slight facial outgrowth was seen at a low frequency. Also, a weak dorsalization phenotype was seen at 2-fold higher MO doses based on a decreased somite size.

To rule out the possibility that the hematopoietic defect observed in the spry4^{MO} was secondary to a vascular defect, spry4 MO was injected into fli1:eGFP Tg zebrafish. While the resulting embryos exhibited minor defects in cardinal vein remodeling and morphogenesis of inter-segmental vessels in the posterior tail, there were no major defects in vascular development (FIG. 4). The integrity of the vascular network in spry4^{MO} fish was further demonstrated by the un-impeded circulation of the remaining DsRed⁺ blood cells in gata1:DsRed Tg spry4^{MO} (data not shown).

Thisse et al. have shown that overexpression of zebrafish spry4 MRNA leads to an expansion of the posterior intermediate cell mass (ICM) [48]. Human SPRY1 was overexpressed in gata1:DsRed Tg zebrafish embryos, and a similar dose-dependent expansion of DsRed⁺ blood cells in the posterior ICM (FIG. 4) was observed. Q-RT-PCR analysis of embryos overexpressing SPRYI revealed a 1.7 and 1.4 fold increase hbae1 and lcp1 mRNA levels, respectively, confirming the observed expansion of blood cells in the ICM. Co-injection of human SPRY1 cDNA with spry4 MOs ameliorated the spry4^{MO} phenotype, yet another confirmation of the specificity of MO targeting (FIG. 4). The similar hematopoietic phenotypes observed following the overexpression of either human SPRY1 or zebrafish spry4 indicate that these genes encode proteins with a similar functional potential in blood development. In addition to the blood phenotype the overexpression often caused a reduction and/or curve in the posterior tail (FIG. 4) not seen in the overexpression of zebrafish spry4 [48]. Thus, overexpression of human SPRY1 causes increased blood and MO rescue. Spry1 overexpression can also inhibit repopulating cells.

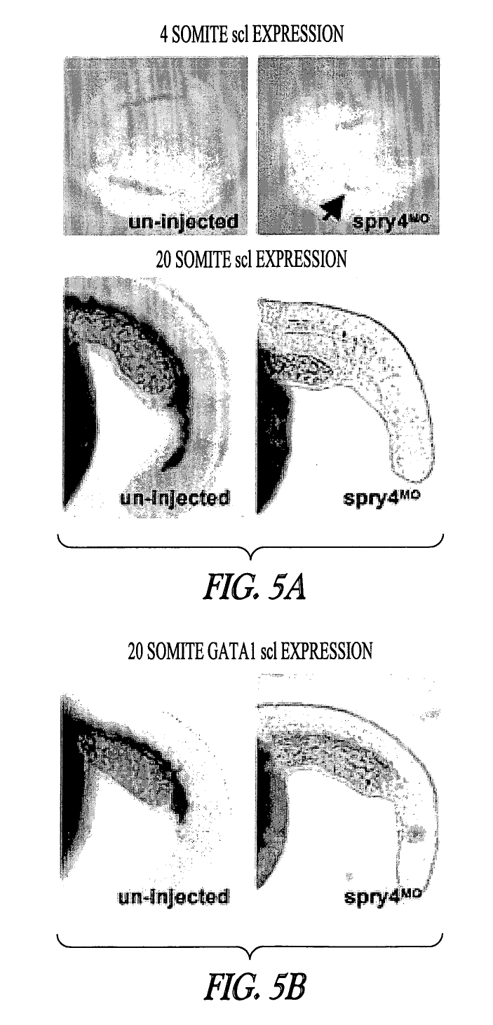

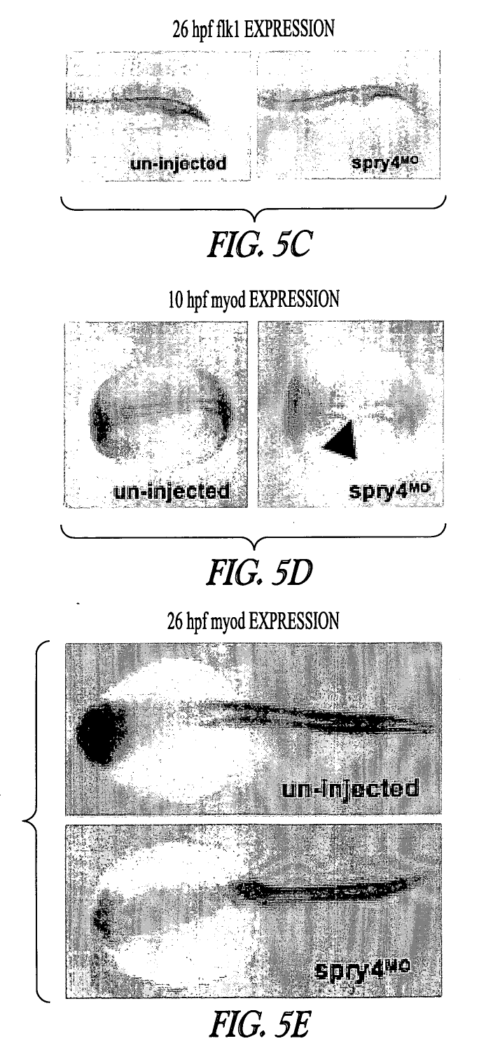

Characterization of hematopoietic gene expression in the spry4^{MO} by whole-mount in situ hybridization revealed a reduction in scl expression at 4 somites (8/15), and virtually no scl (12/20) or gata1 (18/25) expression at 20 somites (FIG. 5), consistent with a defect in mesodermal commitment to HSCs and/or HSC proliferation and differentiation. The few hematopoietic cells that are present in the morphant are hemoglobinized based on o-dianisidine staining (data not shown). To determine whether the hematopoietic defects observed in spry4^{MO} were the result of a defect in mesoderm specification during development, whole-mount in situ hybridization was performed for the vasculature-specific flk1 and muscle-specific myod (GeneID: 30513) transcripts. At 10 hours post-fertilization (hpf) there was a slight defect in myod expression (5/10), while myod expression at 26 hpf was comparable to wildtype (21/21) (FIG. 5). This suggests that there were no major defects in mesodermal commitment in the spry4 morphants. Considering the nearly absent expression of the early hemangioblast and hematopoietic stem cell marker scl, these results suggest that the defect induced by the spry4^{MO} occurs prior to HSC specification. However, the normal expression pattern of other mesodermal genes such as flk1 and myod at 26 hpf in the spry4^{MO} (FIG. 5), indicate that the hematopoietic phenotype is not merely a consequence of defective specification of mesoderm. Finally, the hematopoietic gene cmyb (GeneID: 30519), a presumed marker of definitive HSC in zebrafish [49], was absent at 38 hpf (10/12) (data not shown), suggesting that the spry4^{MO} embryos are devoid of definitive HSC.

In vertebrates, Sprouty family members act as antagonists for fibroblast growth factor (FGF), vascular endothelial growth factor and epidermal growth factor signaling, and they may be involved in feedback regulation, as Sprouty gene expression is induced by activation of these signaling pathways [50]. Sprouty genes antagonize receptor tyrosine kinase (RTK) signaling at the level of the Ras/Raf/MAPK (Mitogen-Activated Protein Kinase) pathway; however, they also can serve as positive regulators of these pathways in some cell types [50]. Therefore, while not wishing to be limited to a particular mechanism, it is believed that SPRY1 affects HSC by modulating FGF-mediated, perhaps in combination with other RTK-mediated, signaling. In fact, three of the 14 genes that induce a hematopoietic defect in the zebrafish screen, SPRY1,MAFB and SPARC, are all involved in FGF signaling [50,51,52], thus suggesting a role for FGF in hematopoiesis.

The sequential genetic screen in zebrafish (optionally followed by confirmation in mammalian models (such as mammalian HSC models)) will establish a hematopoietic function for genes identified by gene array analysis in a high-throughput and efficient manner.

Genes, or the proteins they code for, can be used to expand (e.g., propagate) stem cells, by using the gene itself, or by using small molecules or siRNA (small interfering RNA (siRNA; SiRNAs usually have a well defined structure: a short (about 21-nt) double-strand of RNA (dsRNA) with 2-nt 3′ overhangs on either end), sometimes known as short interfering RNA or silencing RNA, are a class of 20-25 nucleotide-long RNA molecules that play a variety of roles in biology. Most notably, this is the RNA interference pathway (RNAi) where the siRNA interferes with the expression of a specific gene, additionally, siRNAs play additional roles in RNAi-related pathways, e.g., as an antiviral mechanism or in shaping the chromatin structure of a genome).

### BIBLIODRAGHY

1 Morrison S J, Uchida N, Weissman I L (1995) The biology of hematopoietic stem cells. Annu Rev Cell Dev Biol 11: 35-71.
2 Krause D S (2002) Regulation of hematopoietic stem cell fate. Oncogene 21: 3262-3269.
3 Sauvageau G, Thorsteinsdottir U, Eaves C J, Lawrence H J, Largrnan C, et al. (1995) Overexpression of HOXB4 in hematopoietic cells causes the selective expansion of more primitive populations in vitro and in vivo. Genes Dev 9: 1753-1765.
4 Antonchuk J, Sauvageau G, Humphries R K (2001) HOXB4 overexpression mediates very rapid stem cell regeneration and competitive hematopoietic repopulation. Exp Hematol 29: 1125-1134.
5 Buske C, Feuring-Buske M, Abramovich C, Spiekermann K, Eaves C J, et al. (2002) Deregulated expression of HOXB4 enhances the primitive growth activity of human hematopoietic cells. Blood 100: 862-868.
6 Vamum-Finney B, Xu L, Brashem-Stein C, Nourigat C, Flowers D, et al. (2000) Pluripotent, cytokine-dependent, hematopoietic stem cells are immortalized by constitutive NotchI signaling. Nat Med 6: 1278-1281.
7 Reya T, Duncan A W, Ailles L, Domen J, Scherer D C, et al. (2003) A role for Wnt signalling in self-renewal of haematopoietic stem cells. Nature 423: 409-414.
8 Murdoch B, Chadwick K, Martin M, Shojaei F, Shah K V, et al. (2003) Wnt-5A augments repopulating capacity and primitive hematopoietic development of human blood stem cells in vivo. Proc Natl Acad Sci U S A 100: 3422-3427.
9 Bhardwaj G, Murdoch B, Wu D, Baker D P, Williams K P, et al. (2001) Sonic hedgehog induces the proliferation of primitive human hematopoietic cells via BMP regulation. Nat Immunol 2: 172-180.
10 Terskikh A V, Miyamoto T, Chang C, Diatchenko L, Weissman I L (2003) Gene expression analysis of purified hematopoietic stem cells and committed progenitors. Blood 102: 94-101.
11 Park I K, He Y, Lin F, Laerum O D, Tian Q, et al. (2002) Differential gene expression profiling of adult murine hematopoietic stem cells. Blood 99: 488-498.
12 Ivanova N B, Dimos J T, Schaniel C, Hackney J A, Moore K A, et al. (2002) A stem cell molecular signature. Science 298: 601-604.
13 Ramalho-Santos M, Yoon S, Matsuzaki Y, Mulligan R C, Melton D A (2002) “Stemness”: transcriptional profiling of embryonic and adult stem cells. Science 298: 597-600.
14 Fortunel N O, Otu H H, Ng H H, Chen J, Mu X, et al. (2003) Comment on “‘Stemness’: transcriptional profiling of embryonic and adult stem cells” and “a stem cell molecular signature”. Science 302: 393; author reply 393.
15 Bhatia M, Wang J C, Kapp U, Bonnet D, Dick J E (1997) Purification of primitive human hematopoietic cells capable of repopulating immune-deficient mice. Proc Natl Acad Sci U S A 94: 5320-5325.
16 Shojaei F, Gallacher L, Bhatia M (2004) Differential gene expression of human stem progenitor cells derived from early stages of in utero human hematopoiesis. Blood 103: 2530-2540.
17 Liu H, Verfaillie C M (2002) Myeloid-lymphoid initiating cells (ML-IC) are highly enriched in the rhodamine-c-kit(+)CD33(−)CD38(−) fraction of umbilical cord CD34(+) cells. Exp Hematol 30: 582-589.
17 Cheng T, Rodrigues N, Shen H, Yang Y, Dombkowski D, et al. (2000) Hematopoietic stem cell quiescence maintained by p21cip1/waf1 . Science 287: 1804-1808.
19 Seidel M G, Look A T (2001) E2A-HLF usurps control of evolutionarily conserved survival pathways. Oncogene 20: 5718-5725.
20 Buonamici S, Chakraborty S, Senyuk V, Nucifora G (2003) The role of EVI1 in normal and leukemic cells. Blood Cells Mol Dis 31: 206-212.
21 Warren A J, Colledge W H, Carlton M B, Evans M J, Smith A J, et al. (1994) The oncogenic cysteine-rich LIM domain protein rbtn2 is essential for erythroid development. Cell 78: 45-57.
22 Zhou Y, Lim K C, Onodera K, Takahashi S, Ohta J, et al. (1998) Rescue of the embryonic lethal hematopoietic defect reveals a critical role for GATA-2 in urogenital development. Embo J 17: 6689-6700.
23 Travis A, Amsterdam A, Belanger C, Grosschedl R (1991) LEF-1, a gene encoding a lymphoid-specific protein with an HMG domain, regulates T-cell receptor alpha enhancer function [corrected]. Genes Dev 5: 880-894.
24 Bigas A, Martin D I, Milner L A (1998) Notch1 and Notch2 inhibit myeloid differentiation in response to different cytokines. Mol Cell Biol 18: 2324-2333.
25 Hashizume H, Hamalainen H, Sun Q, Sucharczuk A, Lahesmaa R (2003) Downregulation of mafb expression in T-helper cells during early differentiation in vitro. Scand J Immunol 57: 28-34.
26 Geiman T M, Muegge K (2000) Lsh, an SNF2/helicase family member, is required for proliferation of mature T lymphocytes. Proc Natl Acad Sci U S A 97: 4772-4777.
27 Birve A, Sengupta A K, Beuchle D, Larsson J, Kennison J A, et al. (2001) Su(z)12, a novel Drosophila Polycomb group gene that is conserved in vertebrates and plants. Development 128: 3371-3379.
28 Ruzinova M B, Benezra R (2003) Id proteins in development, cell cycle and cancer. Trends Cell Biol 13: 410-418.
29 Hoyle C F, Sherrington P, Hayhoe F G (1988) Translocation (3;6)(q21;p21) in acute myeloid leukemia with abnormal thrombopoiesis and basophilia. Cancer Genet Cytogenet 30: 261-267.
30 Sonoki T, Harder L, Horsman D E, Karran L, Taniguchi I, et al. (2001) Cyclin D3 is a target gene oft(6;14)(p21.1;q32.3) of mature B-cell malignancies. Blood 98: 2837-2844.
31 Nagarajan L, Louie E, Tsujimoto Y, ar-Rushdi A, Huebner K, et al. (1986) Localization of the human pim oncogene (PIM) to a region of chromosome 6 involved in translocations in acute leukemias. Proc Natl Acad Sci U S A 83: 2556-2560.
32 Ekker S C (2000) Morphants: a new systematic vertebrate functional genomics approach. Yeast 17: 302-306.
33 Ackermann G E, Paw B H (2003) Zebrafish: a genetic model for vertebrate organogenesis and human disorders. Front Biosci 8: d1227-1253.
34 Liao W, Ho C Y, Yan Y L, Postlethwait J, Stainier D Y (2000) Hhex and sc1 function in parallel to regulate early endothelial and blood differentiation in zebrafish. Development 127: 4303-4313.
35 Thisse C, Zon L I (2002) Organogenesis--heart and blood formation from the zebrafish point of view. Science 295: 457-462.
36 Nasevicius A, Ekker S C (2000) Effective targeted gene ‘knockdown’ in zebrafish. Nat Genet 26: 216-220.
37 Galloway J L, Wingert R A, Thisse C, Thisse B, Zon L I (2005) Loss of gata1 but not gata2 converts erythropoiesis to myelopoiesis in zebrafish embryos. Dev Cell 8: 109-116.
38 Haffter P, Granato M, Brand M, Mullins M C, Hammerschmidt M, et al. (1996) The identification of genes with unique and essential functions in the development of the zebrafish, Danio rerio. Development 123: 1-36.
39 Pickart M A, Sivasubbu S, Nielsen A L, Shriram S, King R A, et al. (2004) Functional genomics tools for the analysis of zebrafish pigment. Pigment Cell Res 17: 461-470.
40 Golling G, Amsterdam A, Sun Z, Antonelli M, Maldonado E, et al. (2002) Insertional mutagenesis in zebrafish rapidly identifies genes essential for early vertebrate development. Nat Genet 31: 135-140.
41 Leung A Y, Mendenhall E M, Kwan T T, Liang R, Eckfeldt C, et al. (2005) Characterization of expanded intermediate cell mass in zebrafish chordin morphant embryos. Dev Biol 277: 235-254.
42 Hammerschmidt M, Pelegri F, Mullins M C, Kane D A, van Eeden F J, et al. (1996) dino and mercedes, two genes regulating dorsal development in the zebrafish embryo. Development 123: 95-102.
43 Kobayashi K, Hernandez L D, Galan J E, Janeway C A, Jr., Medzhitov R, et al. (2002) IRAK-M is a negative regulator of Toll-like receptor signaling. Cell 110: 191-202.
44 Forster R, Schubel A, Breitfeld D, Kremmer E, Renner-Muller I, et al. (1999) CCR7 coordinates the primary immune response by establishing functional microenvironments in secondary lymphoid organs. Cell 99: 23-33.
45 Gilmour D T, Lyon G J, Carlton M B, Sanes J R, Cunningham J M, et al. (1998) Mice deficient for the secreted glycoprotein SPARC/osteonectin/BM40 develop normally but show severe age-onset cataract formation and disruption of the lens. Embo J 17: 1860-1870.
46 Chida K, Hara T, Hirai T, Konishi C, Nakamura K, et al. (2003) Disruption of protein kinase Ceta results in impairment of wound healing and enhancement of tumor formation in mouse skin carcinogenesis. Cancer Res 63: 2404-2408.
47 Brun A C, Bjomsson J M, Magnusson M, Larsson N, Leveen P, et al. (2004) Hoxb4-deficient mice undergo normal hematopoietic development but exhibit a mild proliferation defect in hematopoietic stem cells. Blood 103: 4126-4133.
48 Furthauer M, Reifers F, Brand M, Thisse B, Thisse C (2001) sprouty4 acts in vivo as a feedback-induced antagonist of FGF signaling in zebrafish. Development 128: 2175-2186.
49 Thompson M A, Ransom D G, Pratt S J, MacLennan H, Kieran M W, et al. (1998) The cloche and spadetail genes differentially affect hematopoiesis and vasculogenesis. Dev Biol 197: 248-269.
50 Kim H J, Bar-Sagi D (2004) Modulation of signalling by Sprouty: a developing story. Nat Rev Mol Cell Biol 5: 441-450.
51 Brekken R A, Sage E H (2001) SPARC, a matricellular protein: at the crossroads of cell-matrix communication. Matrix Biol 19: 816-827.
52 Marin F, Charnay P (2000) Hindbrain patterning: FGFs regulate Krox20 and mafB/kr expression in the otic/preotic region. Development 127: 4925-4935.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth wherein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the statements. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the statements. As used herein and in the appended statements, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as presented in the statements. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended statements.

What is claimed is:

1. An in vivo method of assigning function to a gene comprising:
a) providing a gene expression profiling dataset;
b) identifying at least one ortholog in an animal model for at least one gene of an unknown function from the dataset;
c) altering expression of the ortholog in the animal model;
d) detecting one or more changes in the animal model due to alteration of the expression; and
e) correlating the one or more changes in the animal model with a function of the gene.

2. The method of claim 1, further comprising compiling a functional profile comprising repeating steps b)-e) until two or more genes from the dataset having unknown function are associated with a function.

3. The method of claim 1, wherein the gene expression profiling dataset is obtained from differential gene expression analysis.

4. The method of claim 3, wherein the differential gene expression analysis is obtained from gene microarray analysis or quantitative PCR analysis.

5. The method claim 1, wherein the animal model is a mouse, rat, zebrafish or xenopus animal model.

6. The method claim 1, wherein the animal model is an embryonic model.

7. The method of claim 1, wherein the expression of the ortholog in the animal is decreased.

8. The method of claim 7, wherein the expression of the ortholog is decreased by an antisense oligonucleotide.

9. The method of claim 8, wherein the antisense oligonucleotide is a morpholino antisense oligonucleotide.

10. The method of claim 1, wherein the one or more changes detected are phenotypic.

11. The method of claim 10, wherein the phenotypic change is an alteration in blood cell production or transcription factor expression.

12. The method claim 1, wherein the expression of the ortholog in the animal is increased.


SEQUENCE LISTING (88 sequences)

SEQ ID NO: 1 - DNA, 25 bp, Artificial Sequence
  A synthetic oligonucleotide
ctgtactttttgccataactctact

SEQ ID NO: 2 - DNA, 25 bp, Artificial Sequence
  A synthetic oligonucleotide
tgtttatgtttatggctcatctaag

SEQ ID NO: 3 - DNA, 25 bp, Artificial Sequence
  A synthetic oligonucleotide
taatttagctcctttcatcctcaga

SEQ ID NO: 4 - DNA, 25 bp, Artificial Sequence
  A synthetic oligonucleotide
gatgatgtcattcctcacctctgca

SEQ ID NO: 5 - DNA, 25 bp, Artificial Sequence
  A synthetic oligonucleotide
ccacccataccttcagctccatgcc

SEQ ID NO: 6 - DNA, 25 bp, Artificial Sequence
  A synthetic oligonucleotide
acccatttggtgttcagtcatgttg

SEQ ID NO: 7 - DNA, 25 bp, Artificial Sequence
  A synthetic oligonucleotide
ggatgcaatggaatgaggaagccat

SEQ ID NO: 8 - DNA, 25 bp, Artificial Sequence
  A synthetic oligonucleotide
taacagcccaagcaaacatcttcgc

SEQ ID NO: 9 - DNA, 25 bp, Artificial Sequence
  A synthetic oligonucleotide
gccagaggcccaccagtcaaatgtg

SEQ ID NO: 10 - DNA, 25 bp, Artificial Sequence
  A synthetic oligonucleotide
ttggcgctgatctagcgattatttt

SEQ ID NO: 11 - DNA, 25 bp, Artificial Sequence
  A synthetic oligonucleotide
ttggcgctgatctagcgattatttt

SEQ ID NO: 12 - DNA, 25 bp, Artificial Sequence
  A synthetic oligonucleotide
ctaggacagaaagcacaagcaaatc

SEQ ID NO: 13 - DNA, 25 bp, Artificial Sequence
  A synthetic oligonucleotide
gcatgacaataaatgaatccagaac

SEQ ID NO: 14 - DNA, 25 bp, Artificial Sequence
  A synthetic oligonucleotide
gtgctgttatccgccatcactccct

SEQ ID NO: 15 - DNA, 25 bp, Artificial Sequence
  A synthetic oligonucleotide
ctcccattcccgcacaagcaagccc

SEQ ID NO: 16 - DNA, 25 bp, Artificial Sequence
  A synthetic oligonucleotide
ctctctcatatccatctctcacctg

SEQ ID NO: 17 - DNA, 25 bp, Artificial Sequence
  A synthetic oligonucleotide
ggctctccctcattgtttgctcaca

SEQ ID NO: 18 - DNA, 25 bp, Artificial Sequence
  A synthetic oligonucleotide
acatcccttcaactccttgtccaac

SEQ ID NO: 19 - DNA, 25 bp, Artificial Sequence
  A synthetic oligonucleotide
gttctccatctgcaagtgtagtatt

SEQ ID NO: 20 - DNA, 25 bp, Artificial Sequence
  A synthetic oligonucleotide
tctgatggaactcatacttgcactt

SEQ ID NO: 21 - DNA, 25 bp, Artificial Sequence
  A synthetic oligonucleotide
cagagtaagaagtactgttaatgac

SEQ ID NO: 22 - DNA, 25 bp, Artificial Sequence
  A synthetic oligonucleotide
catgctgccatccttcattccctag

SEQ ID NO: 23 - DNA, 25 bp, Artificial Sequence
  A synthetic oligonucleotide
tttgacaatcccactcatttcagct

SEQ ID NO: 24 - DNA, 25 bp, Artificial Sequence
  A synthetic oligonucleotide
catctcagaacggtttcagttgatc

SEQ ID NO: 25 - DNA, 25 bp, Artificial Sequence
  A synthetic oligonucleotide
gcgtctgacatggtggtagttgaat

SEQ ID NO: 26 - DNA, 25 bp, Artificial Sequence
  A synthetic oligonucleotide
tacatggctgcgtggacaatagtaa

SEQ ID NO: 27 - DNA, 25 bp, Artificial Sequence
  A synthetic oligonucleotide
ccaggtgtgaatgttatgatgtttg

SEQ ID NO: 28 - DNA, 25 bp, Artificial Sequence
  A synthetic oligonucleotide
gagccatcctaaaatagcgttcgtg

SEQ ID NO: 29 - DNA, 25 bp, Artificial Sequence
  A synthetic oligonucleotide
atattgtgtcataggcaaacttctg

SEQ ID NO: 30 - DNA, 25 bp, Artificial Sequence
  A synthetic oligonucleotide
cgctcatcgtgcccctgttcttcag

SEQ ID NO: 31 - DNA, 25 bp, Artificial Sequence
  A synthetic oligonucleotide
tcggaccctagtcttgataagcagt

SEQ ID NO: 32 - DNA, 25 bp, Artificial Sequence
  A synthetic oligonucleotide
tgaccagaccttcaacacattcacc

SEQ ID NO: 33 - DNA, 25 bp, Artificial Sequence
  A synthetic oligonucleotide
aagtttccgttgccatttctggtgc

SEQ ID NO: 34 - DNA, 25 bp, Artificial Sequence
  A synthetic oligonucleotide
ggctcttttccttcgtctccatcag

SEQ ID NO: 35 - DNA, 25 bp, Artificial Sequence
  A synthetic oligonucleotide
ggcttccaaacaaaaagctcatctc

SEQ ID NO: 36 - DNA, 25 bp, Artificial Sequence
  A synthetic oligonucleotide
atatcaagctaaaatgtgtctcaga

SEQ ID NO: 37 - DNA, 25 bp, Artificial Sequence
  A synthetic oligonucleotide
agctgccatagtaactgatcacctg

SEQ ID NO: 38 - DNA, 25 bp, Artificial Sequence
  A synthetic oligonucleotide
aagcgactctctcaccctcggcttg

SEQ ID NO: 39 - DNA, 25 bp, Artificial Sequence
  A synthetic oligonucleotide
gagtcacagtgcaccagagactcag

SEQ ID NO: 40 - DNA, 25 bp, Artificial Sequence
  A synthetic oligonucleotide
gctcggttttaccagccatgattct

SEQ ID NO: 41 - DNA, 25 bp, Artificial Sequence
  A synthetic oligonucleotide
ggcacccgacatgctgagagatttg

SEQ ID NO: 42 - DNA, 25 bp, Artificial Sequence
  A synthetic oligonucleotide
ttcgcattaacttgtaggtcgccat

SEQ ID NO: 43 - DNA, 25 bp, Artificial Sequence
  A synthetic oligonucleotide
tttgcacgagcctggacactggagt

SEQ ID NO: 44 - DNA, 25 bp, Artificial Sequence
  A synthetic oligonucleotide
gtacactcataatgatgcttctcag

SEQ ID NO: 45 - DNA, 25 bp, Artificial Sequence
  A synthetic oligonucleotide
gagccattctcaacaaactcctctg

SEQ ID NO: 46 - DNA, 25 bp, Artificial Sequence
  A synthetic oligonucleotide
gcgaccgctctttacctttcatgtt

SEQ ID NO: 47 - DNA, 25 bp, Artificial Sequence
  A synthetic oligonucleotide
tgacactcatcttgtgcgtaaactt

SEQ ID NO: 48 - DNA, 25 bp, Artificial Sequence
  A synthetic oligonucleotide
tatagttccaacaggcaaacttaat

SEQ ID NO: 49 - DNA, 25 bp, Artificial Sequence
  A synthetic oligonucleotide
ctcatgtttgaagcccccgaaggac

SEQ ID NO: 50 - DNA, 25 bp, Artificial Sequence
  A synthetic oligonucleotide
caaggcaaattctccagacatccta

SEQ ID NO: 51 - DNA, 25 bp, Artificial Sequence
  A synthetic oligonucleotide
aatttatatgaacgcaaaatatgtg

SEQ ID NO: 52 - DNA, 25 bp, Artificial Sequence
  A synthetic oligonucleotide
cgttgtcgttgaaaccaagtgtcgg

SEQ ID NO: 53 - DNA, 25 bp, Artificial Sequence
  A synthetic oligonucleotide
ggctgtccattgtgcgttttttatc

SEQ ID NO: 54 - DNA, 25 bp, Artificial Sequence
  A synthetic oligonucleotide
acgtcatgttcaggagatatttcgc

SEQ ID NO: 55 - DNA, 25 bp, Artificial Sequence
  A synthetic oligonucleotide
atcttgagtttcagccttctgtccg

SEQ ID NO: 56 - DNA, 25 bp, Artificial Sequence
  A synthetic oligonucleotide
actccatctgtaggtcgtgttcctc

SEQ ID NO: 57 - DNA, 25 bp, Artificial Sequence
  A synthetic oligonucleotide
gctcagcatttagcactcttgcatt

SEQ ID NO: 58 - DNA, 25 bp, Artificial Sequence
  A synthetic oligonucleotide
ccaatctcccatgtcaacattcagc

SEQ ID NO: 59 - DNA, 24 bp, Artificial Sequence
  A synthetic oligonucleotide
cgattcagacgctcattttcactt

SEQ ID NO: 60 - DNA, 25 bp, Artificial Sequence
  A synthetic oligonucleotide
gaccttaaccagtgctccacctccc

SEQ ID NO: 61 - DNA, 25 bp, Artificial Sequence
  A synthetic oligonucleotide
catctttactgtggagtgttggcaa

SEQ ID NO: 62 - DNA, 25 bp, Artificial Sequence
  A synthetic oligonucleotide
tgcagccgaaatcacaggaacgtaa

SEQ ID NO: 63 - DNA, 25 bp, Artificial Sequence
  A synthetic oligonucleotide
gacactccatgaatctgtgagctag

SEQ ID NO: 64 - DNA, 25 bp, Artificial Sequence
  A synthetic oligonucleotide
ggctgtggtcatcttccaatcgttg

SEQ ID NO: 65 - DNA, 20 bp, Homo sapiens
ttccaaaggttgggactctg

SEQ ID NO: 66 - DNA, 20 bp, Homo sapiens
tggaggtgatggcttctttt

SEQ ID NO: 67 - DNA, 20 bp, Homo sapiens
cacaggggaggtgatagcat

SEQ ID NO: 68 - DNA, 20 bp, Homo sapiens
ctcaagttgggggacaaaaa

SEQ ID NO: 69 - DNA, 20 bp, Homo sapiens
aacggttctttgaccagcac

SEQ ID NO: 70 - DNA, 20 bp, Homo sapiens
tgtggcgtggtatgctgtat

SEQ ID NO: 71 - DNA, 20 bp, Homo sapiens
cacggcttcactgaggtgta

SEQ ID NO: 72 - DNA, 20 bp, Homo sapiens
agaagtcctctcccccacat

SEQ ID NO: 73 - DNA, 20 bp, Homo sapiens
agccccctctagattcttgg

SEQ ID NO: 74 - DNA, 20 bp, Homo sapiens
ttgtcacacagcagcacaaa

SEQ ID NO: 75 - DNA, 21 bp, Homo sapiens
gcagccgttacacagaaagtc

SEQ ID NO: 76 - DNA, 20 bp, Homo sapiens
cgtcatacgtggcttatgga

SEQ ID NO: 77 - DNA, 22 bp, Homo sapiens
ttcttggatacaggctgatgtg

SEQ ID NO: 78 - DNA, 20 bp, Homo sapiens
ccaatctctccccatgaaaa

SEQ ID NO: 79 - DNA, 20 bp, Homo sapiens
ggaaaggagagggactctgg

SEQ ID NO: 80 - DNA, 20 bp, Homo sapiens
gagccagtgtttctccgaac

SEQ ID NO: 81 - DNA, 20 bp, Homo sapiens
gagctggagaaagccttgaa

SEQ ID NO: 82 - DNA, 20 bp, Homo sapiens
gtctgcaccctttttcctga

SEQ ID NO: 83 - DNA, 20 bp, Danio rerio
tgctggtattgctctcaacg

SEQ ID NO: 84 - DNA, 20 bp, Danio rerio
aacagcaaaggggtcacatc

SEQ ID NO: 85 - DNA, 20 bp, Danio rerio
ttaatgccggactcctgaac

SEQ ID NO: 86 - DNA, 20 bp, Danio rerio
aacaaagtggccagaaccac

SEQ ID NO: 87 - DNA, 20 bp, Danio rerio
cctgacggatgaagagaagc

SEQ ID NO: 88 - DNA, 20 bp, Danio rerio
agacgaccagtggaatggag